(19) United States
(12) Patent Application Publication
(10) Pub. No.: US 2003/0077681 A1
(43) Pub. Date: Apr. 24, 2003

(54) PLK3 protein-protein interactions

(76) Inventor: John P. Cogswell, Durham, NC (US)

Correspondence Address: DAVID J LEVY, CORPORATE INTELLECTUAL PROPERTY, GLAXOSMITHKLINE, FIVE MOORE DR., PO BOX 13398, RESEARCH TRIANGLE PARK, NC, 27709-3398, US

(21) Appl. No.: 10/108,580

(22) Filed: Mar. 28, 2002

Related U.S. Application Data
(60) Provisional application No. 60/284,176, filed on Apr. 17, 2001.

(51) Int. Cl. G01N 33/53; C12Q 1/48; C12N 9/12

(52) U.S. Cl. 435/15; 435/194; 435/7.1

(57) ABSTRACT

Methods of identifying specific protein-protein interactions involving Polo-like kinase 3 are described, and proteins that bind to Polo-like kinase 3 are identified. Methods of screening compounds for the ability to inhibit or enhance such protein-protein interactions are described.

### RELATED APPLICATIONS

The present invention claims priority from U.S. Provisional Application No. 60/284,176, filed Apr. 17, 2001.

### FIELD OF THE INVENTION

The present invention is related to the identification of specific protein-protein interactions, to methods of screening compounds for the ability to inhibit or enhance such interactions, and to methods of affecting physiologic pathways by the inhibition or enhancement of such interactions.

### BACKGROUND OF THE INVENTION

The identification of specific protein-protein interactions assists in understanding the function of specific proteins. Studies of inter-protein reactions define cellular interactions involved in basic biological processes, including the assembly of macromolecular complexes, signal transduction and primary/secondary metabolism, and assist in the identification of novel drug targets and/or biopharmaceutical agents. Additionally, the identification of protein-protein interactions allows the development of screening methods to identify compounds with pharmacological activity (e.g., the ability to inhibit or enhance specific protein-protein interactions).

The yeast two-hybrid assay permits analysis of protein-protein interactions in an intracellular setting, and can screen for large numbers of potential protein-protein interactions. Fields et al., Nature 340:245 (1989); Gyuris et al., Cell 75:791 (1993). The assay utilizes a protein of interest (the “bait”) which is fused to the DNA binding domain of a transcription factor, and a library of target proteins (the “prey”), each of which is fused to a transcriptional activation domain. When a bait protein interacts with a prey protein, a functional transcription factor is reconstituted; the transcription factor activates a reporter gene controlled by a promoter bearing the cognate DNA binding domain site. Detection of the reporter gene product indicates a bait-prey interaction has occurred.

Each bait analyzed using a conventional yeast two-hybrid assay requires retransformation and selection of the prey library. As an alternative, Bendixen et al., Nuc. Acid Res. 22:1778 (1994) have described an interaction mating strategy, in which the prey library is transformed into a haploid yeast strain and then mated with a strain expressing the bait. This strategy permits re-use of the library containing yeast strain for multiple assays. The use of higher-throughput yeast two-hybrid systems has facilitated the ability to map the interactions of collections of related proteins. See, e.g., Bartel et al. (Nature Genetics, 12:72 (1996).

A semi-automated version of the yeast two-hybrid assay originally described by Gyuris et al (Cell 1993, 75:791-803) has been developed (Buckholz et al., J. Molec. Microbiol. Biotechnol., 1:135 (1999)). This system was used to study the interactions of a bait protein with various prey protein libraries. Novel protein-protein interactions were identified, leading to methods of screening compounds for novel pharmacologic activities.

### SUMMARY OF THE INVENTION

A first aspect of the present invention is a method of screening a test compound for the ability to inhibit binding of Polo-like kinase 3 to a pre-selected interactor protein. The interactor protein is selected from among proteins listed in Table 1 herein; proteins comprising an amino acid sequence selected from the interactor sequences disclosed herein; and fragments of such proteins, where the fragment comprises a Polo-like kinase 3 binding site. The method includes selecting an interactor protein and detecting whether the test compound inhibits binding of the interactor and Polo-like kinase 3, compared to binding that would occur in the absence of the test compound.

A further aspect of the present invention is a method of screening a test compound for the ability to bind Polo-like kinase 3 at the binding site for a pre-selected interactor protein. The method includes contacting the test compound with Polo-like kinase 3 (or with a portion of Polo-like kinase 3 containing the appropriate binding site); and then adding the interactor protein and detecting whether the test compound inhibits the binding of the two proteins, compared to that which would occur in the absence of the test compound.

A further aspect of the present invention is a method of identifying a compound which interferes with the binding of Polo-like kinase 3 to a pre-selected protein, where the method comprises forming a mixture of a labeled first protein and a second protein, where one protein is Polo-like kinase 3 (or a binding fragment thereof) and the other protein is a protein that binds to Polo-like kinase 3. The test compound is added to the mixture, and the quantity of the first protein which is bound to the second protein before and after this adding step is determined. A decrease in the quantity of the first protein which is bound to the second protein after the adding step indicates that the test compound interferes with the binding of the two proteins.

A further aspect of the present invention is a method of screening allelic variants of Polo-like kinase 3 for altered protein binding. The method comprises comparing the binding of allelic variants of Polo-like kinase 3 to a pre-selected interactor protein.

A further aspect of the present invention is a method of inhibiting a physiologic pathway, where the pathway includes the step of Polo-like kinase 3 binding to an interactor protein. The method comprises inhibiting the binding of Polo-like kinase 3 to the interactor protein.

### DETAILED DESCRIPTION

Automated Yeast two Hybrid System

In the version of the yeast two-hybrid system described by Gyuris et al., (Cell 1993, 75:791-803), the bait protein is fused to the carboxyl-terminus of the bacterial LexA protein containing the LexA operator-DNA binding domain (DBD). The lexA operator's cognate DNA binding element is incorporated upstream of both a selectable LEU2 reporter gene integrated into the yeast genome, and the lacZ gene on an autonomously replicating plasmid. Prey genes are cloned as either random sequences or cDNAs fused to the carboxyl-terminus of an acid blob transcription activation domain (AD), B42. Association of the AD-prey fusion with the DBD-bait reconstitutes a functional transcription factor, resulting in expression of the LEU2 and lacZ reporter genes. See, e.g., U.S. Pat. No. 5,283,173 to Fields et al., U.S. Pat. No. 5,580,736 to Brent et al. (All US patents cited herein are intended to be incorporated by reference herein in their entirety)

The present inventors utilized an automated format for screening yeast two-hybrids for protein-protein interactions, which includes a liquid array in which pooled library subsets of yeast, expressing up to 1000 different cDNAs, are mated to a yeast strain of the opposite mating type that express the bait protein. See Buckholz et al., J. Molec. Microbiol. Biotechnol. 1:135 (1999); PCT publication No. WO 99/49294, 30 September 1999. Proteins that interact (“interactors”) are detected by assaying for β-galactosidase following prototrophic selection.

The yeast two hybrid (Y2H) assay is carried out in microtiter plates and is partially automated using liquid handling robots. Arrayed prey libraries consist of approximately 1,000 independent pools of 1,000 cDNA clones fused to the BN42 transcriptional activation domain gene. Arrayed libraries are frozen in microtiter plates, and sets of aliquots are thawed as needed. Thawed prey library yeast are mated in microtiter plates with yeast containing bait genes fused to the LexA DNA binding domain. Expression of LEU2 and lacZ are used as reporters for bait-prey interaction in the resulting diploids; cells harboring interactors are selected in media lacking leucine and then tested for β-galactosidase activity.

DNAs encoding interactors are recovered by PCR and sequenced. Any suitable method of sequencing the interactors may be used; in some cases only a portion of the interactor cDNA will be sequenced, as the use of comprehensive cDNA databases such as GenBank allows the identification of an expressed sequence tag (EST) from an analysis of a portion of the EST. See, e.g., published Patent Cooperation Treaty application WO 0015833 (Burns and Weiner, PCT Application No. PCT/US99/21092). Interactor DNA sequences are processed using an automated sequence analysis program, and compared against several genetic databases to identify interactors.

The Bait Protein

The polo-like kinases (Plks) are a family of conserved serine/threonine kinases found in organisms ranging from yeast to humans; the Plk3 serine/threonine kinase is a mammalian member of the family (Ouyang et al., J. Biol. Chem. 272:28646 (1997)). The Plks play a role in normal cell mitosis (Nigg, Curr. Opin. Cell Biol. 10:776 (1998); Glover et al., Genes Dev.12:777 (1998)). At least three Plks have been identified in mammals (Plk1, Plk2 and Plk3). The Plks have been implicated in the origination or progression of tumors. Plk3 has been suggested as a candidate tumor suppressor (Dai et al., Genes Chromosomes Cancer 27:332 (2000); Li et al, J. Biol. Chem. 271:19402 (1996)).

Overexpression of Plk3 in mammalian cells suppresses proliferation and inhibits colony formation, and induces chromatin condensation and apoptosis. Plk3 localizes to the cellular cortex and to the cell midbody during exit from mitosis, and it has been suggested that overexpression or ectopic suppression of Plk3 interferes with cellular proliferation by impeding cytokinesis (Conn et al., Cancer Research 60:6826 (2000)).

It will be appreciated that the term “Polo-like kinase 3” includes naturally occurring allelic variants of the protein; and includes shortened proteins or peptides wherein one or more amino acid is removed from either or both ends of the full-length protein, or from an internal region of the protein, yet the resulting molecule retains activity similar to the full-length protein. The term “Polo-like kinase 3” also includes lengthened proteins or peptides wherein one or more amino acid is added to either or both ends of the protein molecule, or to an internal location in the protein, yet the resulting molecule retains activity similar to the full-length protein.

Polo-like kinase 3 used in the present methods is preferably of mammalian origin, including of human origin. The screening methods of the present invention are useful in identifying compounds with pharmacologic activity of potential use in veterinary and/or human therapeutics.

As used herein, “Polo-like kinase 3” further refers to a protein having an amino acid sequence encoded by SEQ ID NO:1 (SEQ ID NO:2, see GenBank Acc. No. U56998), and to proteins having substantial sequence similarity thereto that retain Polo-like kinase 3 function. “Substantial sequence similarity” between proteins means at least approximately 90% sequence similarity between the amino acid residue sequences, preferably at least approximately 95%, and more preferably at least approximately 97% or 98% similarity.

The phrases “percent identity” or “sequence similarity” refer to the percentage of sequence similarity found in a comparison of two or more amino acid or nucleic acid sequences. Percent identity can be determined electronically, e.g., by using the MegAlign™ program (DNASTAR, Inc., Madison Wis.). The MegAlign™ program can create alignments between two or more sequences according to different methods, e.g., the clustal method. (See, e.g., Higgins, D. G. and P. M. Sharp (1988) Gene 73: 237-244.) The clustal algorithm groups sequences into clusters by examining the distances between all pairs. The clusters are aligned pairwise and then in groups. The percentage similarity between two amino acid sequences, e.g., sequence A and sequence B, is calculated by dividing the length of sequence A, minus the number of gap residues in sequence A, minus the number of gap residues in sequence B, into the sum of the residue matches between sequence A and sequence B, times one hundred. Gaps of low or of no similarity between the two amino acid sequences are not included in determining percentage similarity. Percent identity between nucleic acid sequences can also be counted or calculated by other methods known in the art, e.g., the Jotun Hein method. (See, e.g., Hein, J. (1990) Methods Enzymol. 183: 626-645.)

Also included in the definition of the term Polo-like kinase 3 are modifications of this protein, its subunits and peptide fragments. Such modifications include substitutions of naturally occurring amino acids at specific sites with other molecules, including but not limited to naturally and non-naturally occurring amino acids. For example, conservative amino acid changes may be made, which although they alter the primary sequence of the protein or peptide, do not normally alter its function. Conservative amino acid substitutions include substitutions within the following groups:

Glycine, alanine;

Valine, isoleucine, leucine;

Aspartic acid, glutamic acid;

Asparagine, glutamine;

Serine, threonine;

Lysine, arginine;

Phenylalanine, tyrosine

Discussion of Invention, Terms

The present research identified multiple non-promiscuous proteins that interact with the specific bait protein(s) described herein. An aspect of the present invention is methods of screening test compounds for a specific pharmacologic activity, i.e., methods of screening test compounds for the ability to enhance or inhibit the specific binding of Polo-like kinase 3 (or a binding portion of Polo-like kinase 3) to a selected interactor protein.

As used herein, the term “selected interactor protein” refers to a protein chosen from among the proteins identified herein as non-promiscuous interactors with Polo-like kinase 3 (or a binding portion of Polo-like kinase 3). Interactor proteins and nucleotide sequences encoding interactor proteins are listed in Tables 1, 2 and 3. It will be apparent to one skilled in the art that the present screening methods may be carried out using proteins that comprise an interactor protein sequence disclosed herein, or that comprise the fragment of the interactor protein that contains the Polo-like kinase 3 binding site. Similarly, the methods may be carried out using a fragment of the interactor protein that contains the Polo-like kinase 3 binding site. Proteins with amino acid sequences that are highly similar to the interactor sequences provided in Tables 1-3, and that contain a functional Polo-like kinase 3 binding site, may further be used in the present methods.

As used herein, a binding portion of Polo-like kinase 3 refers to a portion or fragment of that protein which is capable of binding a selected interactor protein, as identified herein. The binding site on Polo-like kinase 3 may be different for different interactor proteins. It will be apparent to those skilled in the art that fragments or portions of the full-length bait protein may possess the same ability to bind an interactor protein as that of the full-length bait protein. The present methods of screening compounds for pharmacologic activity may be carried out using full-length bait protein, or a fragment or portion of the bait protein which is capable of binding the selected interactor protein being used in the screening method. Further, the present methods may be carried out using a protein comprising the fragment of bait protein that binds the selected interactor protein, or comprising the complete bait protein amino acid sequence.

As used herein, a compound that inhibits the interaction (binding) between a bait and interactor protein is one that decreases the ability of the two proteins to bind, either by directly competing with one of the proteins for a binding site on the other protein, or by indirectly inhibiting the binding event. Inhibition need not be complete, as a decrease or reduction in binding may occur. The decrease in binding or interaction of the two proteins is measured in comparison to that which would occur in the absence of the test compound. A compound that competes with an interactor protein and binds to a bait protein may act as either an agonist or antagonist, i.e., it may either mimic the physiologic effects of the binding event, or prevent (completely or partially) the physiologic effects of the binding event. Such compounds may be partial agonists, partial antagonists, or mixed agonist/antagonists.

The decrease or reduction in binding may be evidenced, e.g., by a decrease in the number of bound pairs created, reduced binding affinity between bound pairs, and/or reduced interaction time between bound pairs. The decrease or reduction in binding may be measured using any suitable technique as is known in the art. Such techniques will be readily apparent to those skilled in the art, e.g., competitive binding assays.

As used herein, a compound that enhances the interaction (or binding) between a bait and interactor protein is one that increases the ability of the two proteins to bind. The increase in binding of the two proteins is measured in comparison to that which would occur in the absence of the test compound. The increase in binding may be evidenced, e.g., by an increase in the number of bound pairs created, increased binding affinity between bound pairs, and/or increased interaction time between bound pairs. The increase in or enhancment of binding may be measured using any suitable technique as is known in the art. Such techniques will be readily apparent to those skilled in the art, e.g., competitive binding assays. The identified compounds may be partial agonists, partial antagonists, or mixed agonist/antagonists.

Stated another way, the present methods screen compounds for the ability to affect (inhibit or enhance) the in vivo or in vitro outcome of the binding event, via the compound's effect on the binding event. Where, e.g., the protein-protein binding event is a rate-limiting step in a physiologic pathway, inhibiting the binding event will likewise inhibit the outcome of the pathway as a whole.

The bait protein and an interactor protein, as defined herein, make up a specific binding pair. The term specific binding pair, as used herein, refers to a pair of molecules which are naturally derived or synthetically produced. One of the pair of molecules has an area on its surface (or a cavity) which specifically binds to, and is therefore defined as complementary with, a particular spatial and polar organisation of the other molecule, so that the pair have the property of binding specifically to each other. Examples of types of specific binding pairs include antigen-antibody, biotin-avidin, hormone-hormone receptor, receptor-ligand, enzyme-substrate, lgG-protein A.

The methods of the present invention may utilize labeled proteins. Various methods of detectably labelling proteins are known in the art (e.g., radiolabeling, enzyme labelling, etc.), and one skilled in the art will be able to identify a suitable method.

Therapeutic Methods

The present research has identified previously unknown protein-protein interactions. Where such interactions are involved in pathologic pathways, inhibition (or enhancement) of the protein-protein interaction may provide desirable therapeutic effects. Thus, where a protein-protein interaction is identified as a target for therapeutic intervention due to its involvement in a pathological pathway, methods of affecting (enhancing or inhibiting) the protein-protein interaction provide novel therapeutic strategies. For example, the compound rapamycin links two proteins into a complex, resulting in an immunomodulatory effect. (Choi et al., Science 1996;273(5272):239). Such methods comprise providing to a subject in need of such treatment an effective amount of a compound capable of affecting (enhancing or inhibiting) the identified protein-protein interaction. The effective amount will vary according to the subject and condition being treated, and the active compound. Methods of determining effective doses of active compounds (e.g., dose response studies) are well known to those in the art.

Vectors

A vector is a DNA molecule, capable of replication in a host organism, into which a gene is inserted to construct a recombinant DNA molecule. (See, e.g., Watson et al., Biotechniques 21:255 (1996)).

For the Yeast-2-Hybrid experiments described herein, DNA encoding the bait protein(s) was first cloned into a vector to create an in-frame fusion with the bacterial LexA rep gene. In-frame fusion was verified by sequencing. Portions of the bait genes, or the full-length bait gene, were utilized. Portions or fragments of the bait genes are useful in investigating specific protein domain associations.

Bait Control Assays

Before screening in the Y2H assay, bait control assays may be conducted. Bait control assays include:

1. Sequence of fusion junction, to ensure that the bait construct is fused in frame with the LexA DNA binding domain;

2. Autoactivation assay, to measure the ability of the LexA-bait fusion protein to activate transcription of the reporter in the absence of any interacting proteins; and/or

3. Repression assay, to measure the ability of the LexA-bait fusion to enter the nucleus and bind to the LexA operators upstream from the assay reporter genes.

Y2H Prey Libraries

Libraries of cDNA were transformed into yeast and arrayed into microwell plates for use in the Y2H assay. For example, arrayed Library L4 combined three cDNA libraries derived from human fetal brain, fetal liver, and testis purchased from Invitrogen Corp., Carlsbad, Calif. Another library suitable for Y2H assay is a macrophage library constructed in a modified pYESTrp2 vector. Other cDNA libraries may be screened using the Y2H methods described herein, as would be apparent to one skilled in the art.

### EXAMPLES

### Example 1

### Materials and Methods

The semi-automated yeast two-hybrid assay method described by Buckholz et al. (J. Molec. Microbiol. Biotechnol. 1:135 (1999)) was used to investigate protein-protein interactions using Polo-like kinase 3 (SEQ ID NO:1) as the bait.

Bait protein was cloned into the pMW101 vector, and various cDNA libraries were assayed. Interactor sequences were identified by assaying for β-galactosidase following prototrophic selection. Insert DNA was recovered from the interactors; these DNAs were sequenced and trimmed to remove vector and poor quality regions.

### Example 2

### Database and Sequence Analysis

The interactor sequences identified in Example 1 were compared against the current version of separate genetic databases using BLASTN (nucleotide level) and BLASTX (amino acid level). The genetic databases included four publicly available databases: GenBank; Unigene Unique; Unigene gene; and nrpep (each accessible via the internet website for the National Center for Biotechnology Information (NCBI).

Interactor sequences were provided identifying nomenclature. For some interactors, DNA was recovered and sequenced more than once to ensure accuracy. For these interactors, multiple nearly identical entries occur in the results; the interactor sequence nomenclature will differ only in the repetition designation. That is, the project number will be preceded by a letter to indicate a repetition, e.g., entries b111.a22.03.04.c05.p6.6 and b111.b22.03.04.c05.p6.6 indicate two repetitions, “a” and “b”. These entries do not represent separate discoveries of the same interactor. Separate discoveries of some interactors may be present in the results database, but have identifiers differing by more than just the sequence repetition designation.

BLAST Analysis

Tracefiles were read using Phred to produce files containing the actual basecalls and information about the quality of the reads. Phred is a base-calling algorithm that examines automated sequencer traces with high sensitivity and probability. See Ewing et al. (1998) Genome Res. 8:175-185; Ewing and Green (1998) Genome Res. 8:186-194. DNA sequence matching vector sequences were crossed out (X), and sequence matching known mammalian repeats and low complexity DNA sequences were masked out (N).

The resulting Y2H interactor sequences were then assembled into “contigs” and “singletons” in a database using Phrap (phragment assembly program; a sequence assembly algorithm developed at the University of Washington). Where possible, interactor sequences were assembled by Phrap into “contigs” (overlapping contiguous DNA sequences) containing multiple interactor sequences. Contigs are consensus groupings of at least partially overlapping sequences. This process provides a number of contigs from the Y2H interactor sequences, provides sequence extension, and indicates that the interactor sequence was found elsewhere in the Y2H database. That is, the interactor was encountered before in Y2H analysis, either with the same bait or another bait. Contig information indicates either multiple, independent detections of the bait's association with the same interactor, or that the bait shares a common interactor with another bait. Contigs including other sequences identified with the same bait indicate that the same interactor protein was identified multiple times as interacting with the bait. Contigs including sequences identified using other baits may suggest links between the function of the bait and the other baits, or may suggest that the interaction with the bait was non-specific.

In contrast to contigs, a “singlet” is a sequence containing a single interactor sequence. That is, this sequence represents an interactor found only once with the bait, and was not found as an interactor for other baits.

Sequences that match known promiscuous interacting proteins were removed from the results. “Promiscuous proteins” are those that have been found to interact with numerous unrelated baits. The BLAST results were searched for the following textual terms, which potentially indicate promiscuous proteins: actin; chaperone; collagen related; cytochrome oxidase; ferritin; heat shock; lamin; mitochondri*; PCNA; prote[oa]som*; ribosom*; rRNA; tRNA; ubiquitin; vimentin; zinc finger protein. In addition, interactor sequences that have been found with more than ten unrelated baits are defined as promiscuous interactors and are removed.

Sequences that align with the complementary, non-coding strand of a sequence in one of the target databases are also not reported in the present results.

BLAST Results

The Y2H sequence assemblies (both contigs and singlets) were compared using BLAST with one or more of the following target databases: UniGene unique, UniGene gene (known genes from the UniGene set), GenBank, nrpep (non-redundant peptide, compared on amino acid level), ESTs from GenBank. These databases contain previously identified and annotated sequences. BLAST stands for Basic Local Alignment Search Tool (see, e.g., Altschul et. al., J. Mol. Evol. 36:290 (1993); Altschul et al., J. Mol. Biol. 215:403 (1990)). Final results included matches with the best BLAST scores, quality values, assemblies and blast output.

BLAST results are shown in Tables 1 and 3. A blank BLAST results cell (no entry in the cell) indicates that the identified interactor did not have any significant sequence similarity to any entry in the sequence databases queried.

Nucleotide sequences encoding the bait and interactors are provided in Table 2.

Additional information on interactors is provided in Table 3.

TABLE 1
BLAST hits of Interactors

| Cluster_ID | Sequence I.D. | vs. Unigene uniq | vs. Unigene gene | vs. gcgnuc | vs. gcgprot |
|---|---|---|---|---|---|
| Contig4097 | seq 3 | X13293 | X13293 | E02254 | 1 |
| | | Human mRNA for | Human mRNA for B- | human ‘B myb’ | MYB-RELATED |
| | | B-myb gene | myb gene | oncogene. | PROTEIN B (B-MYB). |
| | | 0 | 0 | 0 | 1E-94 |
| Contig4098 | seq 4 | U01038 | X73458 | X73458 | PLK1_HUMAN |
| | | Human pLK | H. sapiens plk-1 | H. sapiens plk-1 | SERINE/THREONINE- |
| | | mRNA, complete | mRNA | mRNA. | PROTEIN KINASE PLK |
| | | cds | 0 | 0 | (EC 2.7.1.-)(PLK-1) |
| | | 0 | | | (SERINE-THREONINE |
| | | | | | PROTEIN KINASE 13) |
| | | | | | (STPK13). |
| | | | | | 0 |
| Contig4099 | seq 5 | X75315 | X75314 | X75314 | X75314 |
| | | H. sapiens seb4B | H. sapiens seb4D | H. sapiens seb4D | H. sapiens seb4D mRNA. |
| | | mRNA | mRNA | mRNA. | 0 |
| | | 0 | 0 | 0 | |
| Contig4100 | seq 6 | AF086904 | AF086904 | AF086904 | Q9UGF0 |
| | | Homo sapiens | Homo sapiens protein | Homo sapiens protein | BA444G7.1 (PROTEIN |
| | | protein kinase Chk2 | kinase Chk2 (CHK2) | kinase Chk2 (CHK2) | KINASE CHK2) |
| | | (CHK2) mRNA, | mRNA, complete cds | mRNA, complete cds. | (FRAGMENT). |
| | | complete cds | 0 | 0 | 1E-36 |
| | | 0 | | | |
| Contig4101 | seq 7 | no hits | no hits | no hits | no hits |
| Contig4103 | seq 8 | S57501 | J04759 | J04759 | PP12_RABIT |
| | | protein phosphatase | Human protein | Human protein | SERINE/THREONINE |
| | | type 1 catalytic | phosphatase I alpha | phosphatase I alpha | PROTEIN |
| | | subunit [human, | subunit (PPPIA) | subunit (PPPIA) | PHOSPHATASE PP1- |
| | | mRNA, 1400 nt] | mRNA, 3′ end | mRNA, 3′ end. | ALPHA 2 CATALYTIC |
| | | 0 | 0 | 0 | SUBUNIT (EC 3.1.3.16) |
| | | | | | (PP-1A). |
| | | | | | 1E-117 |
| Contig4104 | seq 9 | AI869704 | no hits | no hits | no hits |
| | | w198g02.x1 Homo | | | |
| | | sapiens cDNA, 3′ | | | |
| | | end 0 | | | |
| Contig4105 | seq 10 | AL117237 | AL117237 | AK000726 | Q9UJI9 |
| | | Novel human gene | Novel human gene | Homo sapiens Cdna | HYPOTHETICAL 105.9 |
| | | mapping to | mapping to | FLJ20719 fis, clone | KDA PROTEIN. |
| | | chomosome 1 | chomosome 1 | HEP17004. | 1E-108 |
| | | 0 | 0 | 0 | |
| Contig4707 | seq 11 | AC002544 | AC002544 | AK000739 | no hits |
| | | Homo sapiens | Homo sapiens | Homo sapiens cDNA | |
| | | Chromosome 16 | Chromosome 16 BAC | FLJ20732 fis, clone | |
| | | BAC clone | clone CIT987SK-A- | HEP08682. | |
| | | CIT987SK-A- | 761H5 | 0 | |
| | | 761H5 | 0 | | |
| | | 0 | | | |
| Contig5000 | seq 12 | no hits | no hits | no hits | DSR2_HUMAN |
| | | | | | DOWN SYNDROME |
| | | | | | CRITICAL REGION |
| | | | | | PROTEIN 2 (LEUCINE |
| | | | | | RICH PROTEIN C21- |
| | | | | | LRP). |
| | | | | | 1E-89 |
| Singlet6481 | seq 13 | AL117589 | AL117589 | AB033062 | no hits |
| | | Homo sapiens | Homo sapiens mRNA; | Homo sapiens mRNA for | |
| | | mRNA; cDNA | cDNA | KIAA1236 protein, | |
| | | DKFZp434N178 | DKFZp434N178 | partial cds. | |
| | | (from clone | (from clone | 1E-97 | |
| | | DKEZp434N178) | DKFZp434N178) | | |
| | | 6E-99 | 5E-99 | | |
| Singlet6482 | seq 14 | AJ132583 | AJ132583 | AJ132583 | no hits |
| | | Homo sapiens | Homo sapiens mRNA | Homo sapiens mRNA for | |
| | | mRNA for | for puromycin | puromycin sensitive | |
| | | puromycin sensitive | sensitive | aminopeptidase, partial. | |
| | | aminopeptidase, | aminopeptidase, | 0 | |
| | | partial | partial | | |
| | | 0 | 0 | | |
| Singlet6484 | seq 15 | D42044 | D42044 | D42044 | Q14700 |
| | | Human mRNA for | Human mRNA for | Human mRNA for | KIAA0090 PROTEIN |
| | | KIAA0090 gene, | KIAA0090 gene, | KIAA0090 gene, partial | (FRAGMENT). |
| | | partial cds | partial cds | cds. | 9E-73 |
| | | 0 | 0 | 0 | |
| Singlet6487 | seq 16 | AF034799 | AF034799 | AF034799 | O75334 |
| | | Homo sapiens liprin- | Homo sapiens liprin- | Homo sapiens liprin- | LIPRIN-ALPHA2. |
| | | alpha2 mRNA, | alpha2 mRNA, | alpha2 mRNA, complete | 2E-59 |
| | | complete cds | complete cds | cds. | |
| | | 0 | 0 | 0 | |
| Singlet6488 | seq 17 | AB028998 | AB028998 | AB028998 | Q9UPS7 |
| | | Homo sapiens | Homo sapiens mRNA | Homo sapiens mRNA for | KIAA1075 PROTEIN |
| | | mRNA for | for KIAA1075 | KIAA1075 protein, | (FRAGMENT). |
| | | KIAA1075 protein, | protein, partial cds | partial cds. | 4E-69 |
| | | partial cds | 0 | 0 | |
| | | 0 | | | |
| Singlet6489 | seq 18 | X66276 | X73114 | X73114 | MYPS_HUMAN |
| | | H. sapiens mRNA for | H. sapiens mRNA for | H. sapiens mRNA for | MYOSIN-BINDING |
| | | skeletal muscle C- | slow MyBP-C | slow MyBP-C. | PROTEIN C, SLOW- |
| | | protein | 0 | 0 | TYPE (SLOW MYBP-C) |
| | | 0 | | | (C-PROTEIN, |
| | | | | | SKELETAL MUSCLE |
| | | | | | SLOW-ISOFORM). |
| | | | | | 2E-83 |
| Singlet6491 | seq 19 | no hits | no hits | no hits | no hits |
| Singlet6492 | seq 20 | no hits | no hits | no hits | no hits |
| Singlet6497 | seq 21 | no hits | no hits | G19371 | no hits |
| | | | | human STS SHGC- | |
| | | | | 17415. | |
| | | | | 1E-86 | |
| Singlet6498 | seq 22 | AL079279 | AL079279 | AL079279 | no hits |
| | | Homo sapiens | Homo sapiens mRNA | Homo sapiens mRNA | |
| | | mRNA full length | full length insert | full length insert cDNA | |
| | | insert cDNA clone | cDNA clone | clone EUROIMAGE | |
| | | EUROIMAGE | EUROIMAGE | 248114. | |
| | | 248114 | 248114 | 0 | |
| | | 0 | 0 | | |
| Contig4563 | seq 23 | X59618 | X59618 | X59618 | 1 |
| | | H. sapiens RR2 | H. sapiens RR2 mRNA | H. sapiens RR2 mRNA | RIBONUCLEOSIDE- |
| | | mRNA for small | for small subunit | for small subunit | DIPHOSPHATE |
| | | subunit | ribonucleotide | ribonucleotide reductase. | REDUCTASE M2 |
| | | ribonucleotide | reductase | 0 | CHAIN (EC 1.17.4.1) |
| | | reductase | 0 | | (RIBONUCLEOTIDE |
| | | 0 | | | REDUCTASE). |
| | | | | | 0 |
| Contig5071 | seq 24 | no hits | no hits | no hits | RS2_HUMAN |
| | | | | | 40S RIBOSOMAL |
| | | | | | PROTEIN S2 (S4) |
| | | | | | (LLREP3 PROTEIN). |
| | | | | | 0 |
| Contig5085 | seq 25 | no hits | no hits | no hits | ENOA_HUMAN |
| | | | | | ALPHA ENOLASE (EC |
| | | | | | 4.2.1.11) (2-PHOSPHO-D |
| | | | | | GLYCERATE HYDRO- |
| | | | | | LYASE) (NON- |
| | | | | | NEURAL ENOLASE) |
| | | | | | (NNE) |
| | | | | | (PHOSPHOPYRUVATE |
| | | | | | HYDRATASE). |
| | | | | | 0 |
| Contig5087 | seq 26 | no hits | no hits | no hits | CIB_HUMAN |
| | | | | | SNK INTERACTING |
| | | | | | PROTEIN 2-28 (SIP2-28) |
| | | | | | (CALCIUM AND |
| | | | | | INTEGRIN-BINDING |
| | | | | | PROTEIN CIB)(KIP). |
| | | | | | 1E-88 |
| Contig5185 | seq 27 | gnl\|UG\|Hs#S5565 | gnl\|UG\|Hs#S5565 | X93334 | NU4M_HUMAN |
| | | gnl\|UG\|Hs#S5565 | gnl\|UG\|Hs#S5565 | X93334 Homo sapiens | NADH-UBIQUINONE |
| | | Human mRNA for | Human mRNA for U1 | mitochondrial DNA, | OXIDOREDUCTASE |
| | | U1 small nuclear | small nuclear RNP- | complete genome. | CHAIN 4 (EC 1.6.5.3). |
| | | RNP-specific C . . . | specific C . . . | 0 | 0 |
| | | 0 | 0 | | |
| Singlet6483 | seq 28 | D21064 | D21064 | D21064 | 1 |
| | | Human mRNA for | Human mRNA for | Human mRNA for | MITOCHONDRIAL |
| | | KIAA0123 gene, | KIAA0123 gene, | KIAA0123 gene, partial | PROCESSING |
| | | partial cds | partial cds | cds. | PEPTIDASE ALPHA |
| | | 0 | 0 | 0 | SUBUNIT PRECURSOR |
| | | | | | (EC 3.4.24.64)(ALPHA- |
| | | | | | MPP)(P-55)(HA1523) |
| | | | | | (KIAA0123). |
| | | | | | 1E-48 |
| Singlet6499 | seq 29 | M69039 | L04636 | I76429 | MA32_HUMAN |
| | | Human pre-mRNA | Homo sapiens pre- | Sequence 1 from | COMPLEMENT |
| | | splicing factor | mRNA splicing factor | U.S. Pat. No. 5691447. | COMPONENT 1, Q |
| | | SF2p32, complete | 2 p32 subunit | 0 | SUBCOMPONENT |
| | | sequence | (SF2p32) mRNA, | | BINDING PROTEIN, |
| | | 0 | complete cds | | MITOCHONDRIAL |
| | | | 0 | | PRECURSOR |
| | | | | | (GLYCOPROTEIN |
| | | | | | GC1QBP)(GC1Q-R |
| | | | | | PROTEIN) |
| | | | | | (HYALURONAN- |
| | | | | | BINDING PROTEIN 1) |
| | | | | | (PRE-MRNA SPLICING |
| | | | | | FACTOR SF2, P32 |
| | | | | | SUBUNIT)(P33) |
| | | | | | 7E-36 |

TABLE 2
Sequences

Sequence No.

| | |
|---|---|
| SEQ ID NO1: | ccgcctccga gtgccttgcg cggacctgag ctggagatgc tggccgggct accgacgtca gaccccgggc gcctcatcac |
| Polo-like | ggacccgcgc agcggccgca cctacctcaa aggccgcttg ttgggcaagg ggggcttcgc ccgctgctac gaggccactg |
| kinase 3 | acacagagac tggcagcgcc tacgctgtca aagtcatccc gcagagccgc gtcgccaagc cgcatcagcg cgagaagatc |
| (Bait) | ctaaatgaga ttgagctgca ccgagacctg cagcaccgcc acatcgtgcg tttttcgcac cactttgagg acgctgacaa |
| | catctacatt ttcttggagc tctgcagccg aaagtccctg gcccacatct ggaaggcccg gcacaccctg ttggagccag |
| | aagtgcgcta ctacctgcgg cagatccttt ctggcctcaa gtacttgcac cagcgcggca tcttgcaccg ggacctcaag |
| | ttgggaaatt ttttcatcac tgagaacatg gaactgaagg tgggggattt tgggctggca gcccggttgg agcctccgga |
| | gcagaggaag aagaccatct gtggcacccc caactatgtg gctccagaag tgctgctgag acagggccac ggccctgaag |
| | cggatgtatg gtcactgggc tgtgtcatgt acacgctgct ctgcgggagc cctccctttg agacggctga cctgaaggag |
| | acgtaccgct gcatcaagca ggttcactac acgctgcctg ccagcctctc actgcctgcc cggcagctcc tggccgccat |
| | ccttcgggcc tcaccccgag accgcccctc tattgaccag atcctgcgcc atgacttctt taccaagggc tacacccccg |
| | atcgactccc tatcagcagc tgcgtgacag tcccagacct gacacccccc aacccagcta ggagtctgtt tgccaaagtt |
| | accaagagcc tctttggcag aaagaagaag agtaagaatc atgcccagga gagggatgag gtctccggtt tggtgagcgg |
| | cctcatgcgc acatccgttg gccatcagga tgccaggcca gaggctccag cagcttctgg cccagcccct gtcagcctgg |
| | tagagacagc acctgaagac agctcacccc gtgggacact ggcaagcagt ggagatggat ttgaagaagg tctgactgtg |
| | gccacagtag tggagtcagc cctttgtgct ctgagaaatt gtatagcttt catgccccca gcggaacaga acccggcccc |
| | cctggcccag ccagagcctc tggtgtgggt cagcaagtgg gttgactact ccaataagtt cggctttggg tatcaactgt |
| | ccagccgccg tgtggctgtg ctcttcaacg atggcacaca tatggccctg tcggccaaca gaaagactgt gcactacaat |
| | cccaccagca caaagcactt ctccttctcc gtgggtgctg tgccccgggc cctgcagcct cagctgggta tcctgcggta |
| | cttcgcctcc tacatggagc agcacctcat gaagggtgga gatctgccca gtgtggaaga ggtagaggta cctgctccgc |
| | ccttgctgct gcagtgggtc aagacggact aggctctcct catgctgttt agtgatggca ctgtccaggt gaacttctac |
| | ggggaccaca ccaagctgat tctcagtggc tgggagcccc tccttgtgac ttttgtggcc cgaaatcgta gtgcttgtac |
| | ttacctcgct tcccaccttc ggcagctggg ctgctctcca gacctgcggc agcgactccg ctatgctctg cgcctgctcc |
| | gggaccgcag cccagcttag gacccaagcc ctgaaggcct gaggcctgtg cctgtcaggc tctggccctt gcctttgtgg |
| | ccttccccct tcctttggtg cctcactggg ggctttgggc cgaatccccc agggaatcag ggaccagctt tactggagtt |
| | gggggcggct tgtcttcgct ggctcctacc ccatctccaa gataagcctg agccttagct cccagctagg gggcgttatt |
| | tatggaccac ttttatttat tgtcagacac ttatttattg ggatgtgagc cccagggggc ctcctcctag gataataaac |
| | aattttgca |
| SEQ ID NO:3 | ATTGGAGCTGGAGAGCCCCTCGCTGACA |
| | TCCACCCCAGTGTGCAGCCAGAAGGTGGTGGGCGACCACACCACTGCACC |
| | GGGACAAGACACCCCTGCACCAGAAACATGCTGCGTTTGTAACCCCAGAT |
| | CAGAAGTACTCCATGGACAACACTCCCCACACGCCAACCCCGTTCAAGAA |
| | CGCCCTGGAGAAGTACGGACCCCTGAAGCCCCTGCCACAGACCCCGCACC |
| | TGGAGGAGGACTTGAAGGAGGTGCTGCGTTCTGAGGCTGGCATCGAACTC |
| | ATCATCGAGGACGACATCAGGCCCGAGAAGCAGAAGAGGAAGCCTGGGCT |
| | GCGGCGGAGCCCCATCAAGAAAGTCCGGAAGTCTCTGGCTCTTGACATTG |
| | TGGATGAGGATATGAAGCTGATGATGTCCACATCTCCCCTCCACTCCCCT |
| | GCTTAATAAACTCTAAAAATCCNGNNGNGAAAAAGGNAANNNNNGAANNN |
| | CAGNCNAAGGGAGCAAGGAAAAGAAAAANNNGCCGCGGGGGGTGTTTTCC |
| | TTTTTTTGCACGGGTAGGGGGTCATCCCCCAAAATGAGGTTGGGTTGGAA |
| | AAAAAAATCCTGCTTAAAACCACAAGAAACTTGTTTCACTTATTAGGAAG |
| | GAAAAGATTAATTAAAATGGCCG |
| SEQ ID NO:4 | GAGGTTCGAGAGACAGGTGAGGTG |
| | GTCGACTGCCACCTCAGTGACATGCTGCAGCAGCGGCACAGTGTCAATGC |
| | CTCCAAGCCCTCGGAGCGTGGGCTGGTCAGGCAAGAGGAGGCTGAGGATC |
| | CTGCCTGCATCCCCATCTTCTGGGTCAGCAAGTGGGTGGACTATTCGGAC |
| | AAGTACGGCCTTGGGTATCAGCTCTGTGATAACAGCGTGGGGGTGCTCTT |
| | CAATGACTCAACACGCCTCATCCTCTACAATGATGGTGACAGCCTGCAGT |
| | ACATAGAGCGTGACGGCACTGAGTCCTACCTCACCGTGAGTTCCCATCCC |
| | AACTCCTTGATGAAGAAGATCACCCTCCTTAAATATTTCCGCAATTACAT |
| | GAGCGAGCACTTGCTGAAGGCAGGTGCCAACATCACGCCGCGCGAAGGTG |
| | ATGAGCTCGCCCGGCTGCCCTACCTACGGACCTGGTTCCGCACCCGCAGC |
| | GCCATCATCCTGCACCTCAGCAACGGCAGCGTGCAGATCAACTTCTTCCA |
| | TGATCACACCAAGCTCATCTTGTGCCCACTGATGGCAGCCGTGACCTACA |
| | TCGACGAGAAGCGGGACTTNCCGCACATACCGNCTGAGTCTNCTGGAGGA |
| | GTACGGCTGCTGA |
| SEQ ID NO:5 | Ttatgctccagcttgtaccgagcttagacatactagtcacggctgcgcagtgtggtgggaattcgaatgcttgggggcg* |
| | tg*gaatgtggtagaagaagcagactgaatttactgacagacaggttagcattaaaagattcacaggatatacgctgcaa |
| | cttcagCGcTacgACTGgaaAGGGGCCTTTGGCCGGCGGCCCCTGTTACCGGCGGCCCCTGTGCGCCTGGGAGCTCCTCC |
| | GGGCTTGAGGAAGCCGCCCACGTGCCCTGATGGAGAAAATGGGACTCCAACAGGAGGCcgtgTCCTCACACCTCAGaCTG |
| | CGCTCACAGCTcgngaGGATCAAGTTACAATAAACAGtccATTAaCttCtTGcttTCAGGTTTCCCTGgagtcaggcatc |
| | tctgcacagtccaggcagcccagggctgcagagggctgtacacccgccacatcacagtgggacacagctgag*actgagt |
| | ggaagcagaaagtcagaagctcatgg*cagactgatgcctatagtagatcatccatgcgcgcagtctaagcgctatgtta |
| | ctt |
| SEQ ID NO:6 | CTCTCACTCCAGCTCTGGGACACTGAGCTCCTTAGAGACAGTGTCCACTCAGGAACTCTATTCTATTCCTGAGGACCAAG |
| | AACCTGAGGACCAAGAACCTGAGGAGCCTACCCCTGCCCCCTgggCTCGATTATGGGCCCTTCAGGATGGATTTGCCAAT |
| | CTTGAATGTGTGaATGACAACTACCggtTtgggagGgacaaaagctgtgaatATTgctttgaTGaaCcactgctgaaaag |
| | aacagataaataccgaacatacagcaagaaacactttcggattttcagggaagtgggtc**taAAAaCttttacattgga |
| | taccttagaaaatacagtggcaatggaaacctttgtaattccagaacttgtagggaaaggaaaacccccctcttttgaat |
| | aaccattctt*aaattgcccttgtacttaa*ccggaaataaagg*tttt*ggcttttttgaaccgaccgggaaaaaacaa |
| | accagtttatctctagggctttaaggaatgaatccttttgtcaaaaaccttttgaatgggcccctttgaaaggaaa |
| SEQ ID NO:7 | AaTTGACGACTGCTGCTGGCACATGGAGCCCCTCTCGCCAATTCCCATTGACCACTGGAACCTGGAGCGGACCGGCCCCC |
| | TGAGCACCAGCAGCCCCAGCCGCAGGATGAACGAGGCCGCCGACAGCCGTGACTGTCGCTCCCCGGGACTCCTGGACACC |
| | ACCCCCATCCGAGGAAGCTGCACTACCCAGAGGAAATTGCAAGAGAAGTCCTCGGGCGCGGGCTCCCTGGGGAATAGCAG |
| | GCCGAGCTTTCTGAATTCGGCTCTGTGGGACGTTTGGGACGGGGAAGAGCAGAGGCCTCCAGAGACCCCTCCTCCGGCCC |
| | AGATGCCAAGCGCTGGTGGAGCTCAGAAGCCCGAAGGGTTAGAGACACCCAAAGGTGCTAATCGGAAGAAGAACTtGCCC |
| | CGAAT |
| SEQ ID NO:8 | CTCTTTCTGGGGGACTATGTggacAGGGGCAAGCAGTCCTTGGAGACCATctggctgCTGCtggCCTATAAGATCAAGTA |
| | CCCCGAGAACTTCTTCcTGCTCCGTGGGAACCACGAGTGTGCCAGCATCAACCGCATCTATGGTTTCTACGATGAGTGCA |
| | AGAGACGCTACAACATCAAACTGTGGAAAACCTTCACTGACTGCTTCAACTGCCTGCCCATCGCGGCCATAGTGGACGAA |
| | AAGATCTTCTGCTGCCACGGAGGCCTGTCCCCGGACCTGCAGTCTATGGAGCAGATTCGGCGGATCATGCGGCCCACAGA |
| | TGTGCCTGAGGAgggcCTGCTGTGTGACCTGCTGtgGTCTGACCCTGACAAGGACGTGCAggGCtgtggcGAGaaCGacc |
| | gtGGCGTCTCtTTTACCTtTGGAGccgaggtggtggccaagtTcctccacaaGCAcgacttggacctcatctgccgAGCA |
| | CAccag*gtggtagaAGAcggctacGAGTtCtttgccaaGcggcag*ctggtgacaCTTTtCtcagc*tcccaaCTA*Ct |
| | gtggcaaggttgacaaatgc*tgcggccatgatg*agtgtgg*acgaga*ccctcatgtgctcttttcagatcctcaagc |
| | cc |
| SEQ ID NO:9 | CCTGGCTCCTACTCCAGGTCCCCCGCGGGGTCCCAGCAGCAATTC*GGCTACTCCCCAGGGCAGCAGCA*GACCCACCCC |
| | CAGGGTTCTCCAAGGACATCTACACCATTTGGATCAGGGCGTGGTAGAGAAAAAAGAATGTCTAATGAGTTGGAAAATTA |
| | TTTCAAGCCTTCAATGCTTGAAGATCCTTGGGCTGGCCTAGAACCAGTATATGTAGTGGATATAAGCCAACAATACAGCA |
| | ATACTCAAACATTCACAGGCAAAAAAGGAAGATACTTTTGTTAACATTTCTGAAATTCAACTGGAAGCTTCATGTGTCAG |
| | GAACATCTTGGACAAAACTTTAAGTTGTGTTGATATAAATTTACCCAAAGATGATGACTTTGATTGGATAATTA*GTAaG |
| | GTCTTTTTgttaTTTTTCA*TcgtaTCAggTA*ttgtTGATATTA*GAGaAAAAAGTAggatAACtt*G*caaCATTTAG |
| | ctCT*GGAAGTAcCTACC*ACaatttagagatttaccgtttc*catatatttaacattnctgg*tacantatgggacatt |
| | gnnctttaatgttttttcaatgttttaaaaataaacatt |
| SEQ ID NO:10 | AGAGAGAGAGAGAGAGGAGAAAGTGAGCT |
| | CAGCGAGTTGGCCGGGTGACACACTGATGAGGGGGTCAAAGGACACTCTG |
| | AGTTAGTGCCCTCGGCACACACAGCGAACAGTGATCATGAAAAGAGTGGG |
| | CTCAATAATTTTCCATAAACTTGCTCAAGATTCCATGCAGTTGCCATACA |
| | GTCTTTGAGGTATGGTCAACCTATAGTAAGTTAGTAAATGTTAAGGGGAG |
| | GAAGAAATGGAAACCTAAACATCTACTGCAATGAAAACCAACAGCCATGT |
| | CAGTAGGAGTAATTCAACCTTCGTTGAACACATGAAATTGAACACACTCT |
| | TGTTTTCCCTGGACCTGGCATCTCCAGGTGTCAACACAGAATTAAGCATC |
| | CATAATTGCTCAAAGTTACCTGGCGCATGATGGGTCTTGGTCTTCTTACA |
| | CTTCTTGGTACTTTTCAATTTCATCCATGTCAACAGCCAAGCCAACACAC |
| | TGTTGCTCCAATATGTAAAAGGCACTTCTGTAGGGCTGGCATGAGTCAGT |
| | CAGTTCAAGACAACCTGAAGGAGTTGAATAACATCTATCCAGTGAGTTCT |
| | GCAAGACTTGANGCTCTTTCTCATCCAGCAGCTCTCTGCTGAGCCTGAAN |
| | AAGTTGAGAAAAAGAAAA |
| SEQ ID NO:11 | NTTTTTTNNNNNAGGCCTCCTAGCTCTGATGATGCGCGATGATCAGTCGC |
| | TTCTCACGAGATTCGGAACGAGGCGGAGAAGTTGGAGCAAGGCTGGCCGA |
| | GAACAGATGAACGGGAGCCCGACTACATGCTAGGGCCACCTAGCGGCGTT |
| | ACTTCCGAGACCACATGGACGGCTACCGCAAAAATTAGACCTTACATGTG |
| | CCGCGGTGGCTACCGCCAGCAGCCGCCTCAGACCGGCCTACTGAGCTCTC |
| | CCACCTCTGCATCCCGCCTGGGCCATCCAACCTTGAAGTCCTAAACCACA |
| | CCTCAGTCACTAAAGGTCTGTTTAAAGTTAAAAAAAAAAAAAAAAAAAAA |
| | AAAACCCCGGGGGGGTTGGTGCTTTTTCCCCAAGGGTTTGGGCAAACCCC |
| | CCAAAAAGGGTGCGGGTTTTTAANNNNNNTNNCCCNCANCCNNNNNATTT |
| | TGCTTTTATTCAACCCCTGGGTTGAAAAGAACATAATAAAATACCCGANC |
| | CTTCCCCGCAAAGAAACACCTTTTCGGGATTTTTCAGGGGAAGGGGGGGC |
| | CCCTAAAAAAACCTCTTTAACATTTGCCTTCCCCTNGAAAAAATCCCCCG |
| | GGGGCCCATTTGAAACCCCCTTTTTAAAAACCCACAACCCTTTGTNAGGG |
| | AAAAAGGAAAAACCCCCCCCCCCCTTTTGAATAAACAATTTTCTTGAAAT |
| | ATGCCCCCGGCCCCCTAACGCAAGAAAAAAAAGGTTTTTGGCCTTTTTTT |
| | GGGACCCCCCCTGGGGAAAAAACCANCCCCTTTTTTCTCCCCAGGCCTTT |
| | AAAGAGAAGAAAAACCTTTTTTGTAAAAAACTTTTTGGAAAGGGGCCCCC |
| | TGGAGAGAA |
| SEQ ID NO:12 | GCGCAAGCCGGCGTGCGGTCCCGCGG |
| | CGCTGCAGTTGTGTCCAGCCGGTCACGGGGCGGGTATGGCGGCCACGTTC |
| | TTCGGAGAGGTGGTGAAGGCGCCGTGCCGAGCTGGGACTXXXXXXXXXXX |
| | XXXXXXXXXXXXXXXXXXXXXXXXXXCGCCCGAGGACAGGGAGGTGCGTC |
| | TGCAGCTGGCGCGGAAGAGGGAAGTGCGGCTCCTTCGAAGACAAACAAAA |
| | ACATCTTTGGAAGTTTCTTTGCTAGAAAAATATCCGTGCTCCAAGTTTAT |
| | AATTGCTATAGGAAATAATGCAGTAGCATTTCTGTCATCATTTGTTATGA |
| | ATTCAGGAGTCTGGGAGGAAGTTGGTTGTGCTAAACTCTGGAATGAATGG |
| | TGTAGAACAACAGACACTACACATCTGTCCTCCACAGAGGCTTTTTGTGT |
| | GTTTTATCATCTAAAATCCAATCCCTCGGTTTTTCTCTGTCAGTGCAGTT |
| | GCTATGTTGCAGAAGATCAACAGTATCAGTGGCTGGAAAAGGTTTTTGGC |
| | TCTTGTCCAAGGAAGAACATGCAGATAACTATTCTCACATGTCGACATGT |
| | TACCGATTATAAAACCTCAGAATCCACCGGCAGCCTTCCTTCTNCTTTNC |
| | TGAGAGN |
| SEQ ID NO:13 | GGGTTGTGGGGGATCTGTGTGGGGT |
| | TCTCAACGCAGATCCATCCTGGGGTCTCCCGGGCGGGGATGGCTGACCTC |
| | GAGTCCCCTCCCTTCCCGAGAACCCGCTCTGTCCCGAGGGCAGCTAACAA |
| | GGGCTGAGCCCCAGGTACAGGTTGCCTCTTCCACGGCAGGAATTTTTACC |
| | AAAACCACAAGCAAAAAACAAAACAGACCACCACGACCAACAACAAAGAT |
| | GGGGGGTAGGGTTTTGTAAAGGTTCTGTTAGGTTCATATTTTTATATCAT |
| | TTTGCCCATAAATGCGGAATTTGCCGTGGGAATTTGAAGACAAATGATCT |
| | ATGTTTTTATGGTTCTCTAGGGAAGGTGTTCTGAGGGCCGTGCTCTCTCC |
| | AGCTGTGGGAGGCCTGCTCCCTCTGGNGGGCACCCTGNGCAGTGTGTGGG |
| | GCCTTTGGAGGCGCTCTTGCCAATGCNACGAGTGTGAGCCTGCAGCGTTG |
| | NACGTCCCGACGAAGCTATACTTCTGAGATCGGCTAGAGAGACGCTGACC |
| | TTGACAATGTTGATACATCTGCTCAGCTTATTGTGATNAGATGCTCATGG |
| | TAAAAAAAAAAATAAAAAC |
| SEQ ID NO:14 | CGGAGTGTNNNNTTTGATXXXXXXXXXXXXXXXXXXXXXXXXXXXXXXXX |
| | XXXXXXXXXXXXXXXXXXXXXXGACCGACCCCTTGGCCAAAAAAAAAAAA |
| | AAAGCAAAAAACAAAAACCTACCCTGTTCTGGGTTTTTTCCTCCCTTTAG |
| | TTCCACCCCCAACCCCCATTCCCTGGTGTCCTTCTTAGAGATGAAGAAAT |
| | AATACGGAAACATCTTTCATAGCCACATTAAATAAGAGAAACTGATATAC |
| | ATTATTTTTTTCTTTTTAAAGATGACTTATAAGAACCCTGAAATTTATAT |
| | AGGTGAGACAATAGAAATAAAAAGATCTTCAGCCAGGCCTTTCTGAAGGA |
| | GTTATTCTGCTAAAAATGGTCTTAGTTGTCTGAAAAGCCAGCTCTTGAAC |
| | CTCTTCACAACAGTATCAACACTGGCTTCTCCCGGTTCATTTTATGCGTG |
| | CGAGAAGTCAGTGGTAACTGCTGCAGGGCTTAATACATTAGTGGTAACTG |
| | GTTTAAAAAACAAAGACTGTAAGCCTGTGTGTGCCACTGTTTGCTTCAAC |
| | AGTATATCCTACTAATAAGCCTCACTATTTAATCCAATGAGTTTTAAATC |
| | TAAATCTCATTCCCTTCTTCTTTCCCTACCTTNTTTTCTTTTGTTCTTAA |
| | AAAAATATTTTGTGTATTTACAGAAATTCATTATTGGGTGGCTTAACGGA |
| | TTCCAG |
| SEQ ID NO:15 | CGGGGCTCTTTTNNNNGATGCCTCCTAGCCTGATGATGTGCGAAATCAGT |
| | CGCCGGTGACGAACTGGAAACTGACGCGCGAACGAGTCTGACCGTGCGTG |
| | GAGCGTTTAAGAGGACACTTGAGCAATGCATAAGCCAGCGCGTAATAGCT |
| | TGCTGGACCGGGGCCAGATGATGTAGGTAGTTCAGCAACGCTATCATTTA |
| | CCGACCTCCATCAGTGCCATGGAGGCCACCATCACCGAACGGGGCATCAC |
| | CAGCCGACACCTGCTGATTGGACTACCTTCTGGAGCAATTCTTTCCCTTC |
| | CTAAGGCTTTGCTGGATCCCCGCCGCCCCGAGATCCCAACAGAACAAAGC |
| | AGAGAGGAGAACTTAATCCCGTATTCTCCAGATGTACAGATACACGCAGA |
| | GCGATTCATCAACTATAACCAGACAGTTTCTCGAATGCGAGGTATCTACA |
| | CAGCTCCCTCGGGTCTGGAGTCCACTTGTTTGGTTGTGGCCTATGGTTTG |
| | GACATTTACCAAACTCGAGTCTACCCATCCAAGCAGTTTGACGTTCTGAA |
| | GGATGACTATGACTACGNTGTAATCAGCAGCGTCCTCTTTGCCTGGTTTT |
| | TGCACCATGATCACTAAGAGACTGCACAGGTCAAAGCTCTGGATCGGGCT |
| | TGCGATAAAGAACAAGACTGTGCCTAAAGTGGGAGCCAGGGAGTGTGGGT |
| | AAATACAAGTCACGTTGAGTTTGTGGATTGTGGAGATTGGGGGGGAAGGC |
| | TAACTAAAACTGGGGAAGATGTGACCTCACCAAACTCTT |
| SEQ ID NO:16 | TGGACAGATAGTCTGATT |
| | ACAGAACAACTAAGGTAATAAGAAGACCAAGGAGAGGCCGCATGGGTGTG |
| | CGAAGAGATGAGCCAAAGGTGAAATCTCTTGCGGATCACGAGTGGAATAG |
| | AACTCAACAGATTGGAGTACTAAGCAGCCACCCTTTTGAAAGTGACACTG |
| | AAATGTCTGATATTGATGATGATGACAGAGAAACAATTTTTAGCTCAATG |
| | GATCTTCTCTCTCCAAGTGGTCATTCCGATGCCCAGACGCTAGCCATGAT |
| | GCTTCAGGAACAATTGGATGCCATCAACAAAGAAATCAGGCTAATTCAGG |
| | AAGAAAAAGAATCTACAGAGTTGCGTGCTGAAGAAATTGAAAATAGAGTG |
| | GCTAGTGTGAGCCTCGAAGGCCTGAATTTGGCAAGGGTCCACCCAGGTAC |
| | CTCCATTACTGCCTCTGTTACAGCTTCATCGCTGGCCAGTTCATCTTCCC |
| | CCAGTGGACACTCAACTCCAAAGCTCACCCCCTCGAAGCCCTGCCAGGGA |
| | AATGGATTCGATGGGAGTCATGACACTTGCAAGGGATCTGAGGAAACATC |
| | NGAGAAAGGATGCCAANTTTTGGAAGAAGATGGTTCGGAAGACAAAGCAA |
| | CAATTAAATGTGAAACTTTTCCTCTTCTACCCCTTAAGCCTTAAAAGGGA |
| | TAAACTTTTCTTTTTCTAACCCAAGAAGCTGAAAGAGTTAATTTTCTTT |
| SEQ ID NO:17 | AGTGCGGCC |
| | TGGGCACCCGCTGCCTCTGCTCTTGCCTGCCTGTGGGCATCACCATGCCC |
| | CGATGCCTGACTACAGCTGCCTGAAGCCACCCAAGGCAGGCGAGGAAGGG |
| | CACGAGGGCTGCTCCTACACCATGTGCCCCGAAGGCAGGTATGGGCATCC |
| | AGGGTACCCTGCCCTGGTGACATACAGCTATGGAGGAGCAGTTCCCAGTT |
| | ACTGCCCAGCATATGGCCGTGTGCCTCATAGCTGTGGCTCTCCAGGAGAG |
| | GGCAGAGGGTATCCCAGCCCTGGTGCCCACTCCCCACGGGCTGGCTCCAT |
| | TTNCCCGGGCAGGCCGGCCTATCCACAATCTAGGAAAGCTGAGGCTACGA |
| | AGATCCCTTACGGAGGGAGGGAGGGGGACAGGGAACCCCATTGGCCTGGG |
| | GCAACCTGGACCTTAAGCAAGGAACCTTTTGGCAATCTGCCAGAAGTCCG |
| | CTTGGAGCCCCGGTGTCCCTGGGAAGGGAAGGGGCCCCCCAAATGGGGGA |
| | ACAAAGAACAAATGTGCTTTGGGGGCTTTCCCCCGAGAAGGCCCCCCAAT |
| | GCCAGGGGGTTTTCGTTAAAGAAGTGGGGTTTGGGGCCCCTTTCACAGCC |
| | CCCTTTGACAAACCAAAAAAGTCCACATCCCCAGGGGGAAAGGGAAAAGA |
| | CCCCCTGGGAGAAAGGGGAAAACCCCGGGGCCCCCCC |
| SEQ ID NO:18 | GTGGAGCGTGAGTGGCGTTA |
| | CGAGTGTGACGGGTCTGAAGATGATGCCAATGTAAAAGGGTGCATGAATG |
| | GGGACGAGATAATTCCTGGGCCATAATCAGCATACCTCCTCACAGTTGAG |
| | GGTAAAAAACACATCTTGATCATAGAGGGAGCAACAAAGGCTGATGCTGC |
| | AGAATATTCAGTAATGACAACAGGAGGACAATCATCTGCTAAACTTAGTG |
| | TTGACTTGAAACCTCTGAAGATTTTGACACCTCTGACTGATCAGACTGTA |
| | AATCTTGGAAAAGAAATCTGCCTGAAGTGTGAAATCTCTGAAAACATACC |
| | AGGAAAATGGACTAAAAATGGCCTACCTGTTCAGGAGAGTGACCGTCTAA |
| | AGGTGGTTCAGAAGGGAAGGATCCACAAGTTAGTGATAGCCAATGCCCTC |
| | ACTGAAGATGAAGGTGATTATGTATTTGCACCTGATGCCTACAATGTTAC |
| | TCTGCCTGCCAAAGTTATGGTATTGATTCTTCTAAGATCATNCTGGATTG |
| | TCTTGATGCTGACAACACCATGACGGTGATTGCAGGAAACAGCTTCGTCT |
| | TGAGATTCCCATTAGCGGAGAACCACTTCCTAAACCATTTGGAAGCCGGG |
| | AAGTAAGGTTCTATTGAAAGGCATGGCCCGGTTAAAAACCGAATTTTAAC |
| | TTGGTTGACCCACTTCTGGCATTGATTATACTGAAGGGTGACTTCTGGTT |
| | TTAC |
| SEQ ID NO:19 | ACATTG |
| | AAAGAAATGCCTTGGGGACATATCAATAACAACGTAACACAGAGCTATTC |
| | TATTGGTTATGAAGGTAGCTATGATGCCTCTGCTGATCTCTTTGATGATA |
| | TTGCTAAAGAAATGGACATTGCAACTGAGATTACCAAAAAATCACAGGAT |
| | ATTTTGTTAAAATGGGGAACATCTTTGGCAGAAAGTCACCCTTCAGAGTC |
| | TGATTTTTCACTGAGATCACTTTCTGAAGACTTCATCCAGCCTTCACAAA |
| | AATTATCCTTGCAAAGCCTATCTGACTCTAGGCATTCAAGAACATGCTCT |
| | CCAACACCTCATTTTCAATCAGATTCAGAATATAATTTTGAAAATAGTCA |
| | AGACTTTGTCCATGTTCACAGTCAACTTCAATTTCAGGGTTCACCAAACA |
| | AGAATTCATGGGATAAACAGAGCTTTAAAAAAACCTGATTTTATCAGATC |
| | TTGATGTAACTATTAAAAAATAAGGATTTTCCTTAAAATGACAACCACAA |
| | GCCACCCAACTGGCCAAAAATTTAAAACACTTACCGGAAATAAGAGGCAA |
| | TCCACCACTGGCGGCCTTCAGGATCATTTAAGAGCCAC |
| SEQ ID NO:20 | AATATACAACATGGCTCGAGC |
| | CCATGCCTGCAGGCGCCACGTCTGCACAAGAGAGAGATGACGACATCATA |
| | TGGACATCCACACTCGCAAAGCAGGTCAGGAGGACTGGCATGCCCCTGTC |
| | TCCCCAGCACCCCATTTGTAGCCTTTTCTCAGGTTGAGTAAATAGTTCTG |
| | TATTAGGAAAGGCCCTCTTGCCTCCACAACTCCTTCCCCACCTTGGTGAC |
| | ATCATTCATCGTGGTTCTGCCACTTCCTAGGAGCCCATGGAGGAGAGGCA |
| | CCAA |
| SEQ ID NO:21 | AGGGGNNNNNCCCTTTTNTTATCCTCCTACTTGAGGATGTGCGAAATTAT |
| | GCCTCTGACGAATTGGAACGAGGGGCTAGGCGTAGATTATGGCGGTCTGT |
| | CAAATCTACTTGGGGAGCAGCTAATTCTGGACGAGTTAGCCGGCCTGCTG |
| | CGAGGCCGCTCATAAAGCTGGGACTCCATGACTTACATCACTTCCACTCC |
| | CTTGCCATCCGAGGTGACATGCCCAATCAGATTGTGCAGATCTTGACCCA |
| | GGATCATGGCATGGAATTAATATGTTGCTTTGGCAACACCAGTTGGGACA |
| | GAAGCCTTCTGCTCTTCAGGGCAAAACAAACCATAGAGACTCATCCAATC |
| | CCTGAATCACTGATAGAAAAAGGGAAAGAAAAGAACAGATTAAGATTCCA |
| | GAAGCAGTGAGATGGGAGGGCANGAAGACCAAGAAAGATATTGAAAGGTT |
| | TTATATTGAGAAATATGTTCATTCTTCTTAATTCCTAACAATCANGCAGC |
| | CGCAAAACCTGCAGGAGCTTTTGGTAAAATGTCCAAGGCACAATATTGGA |
| | AAGAATCATAATCTGGTCCCCAATGGTTTTGAACCAAACCTTGAAGAAGA |
| | AGTGAAATCGTGGGGAGGTGAATGAGACCCTAGGGAAATCTCTGGAAATG |
| | GGGAAAAGGGCCCATAGGGAAAAAAGGGGGGCCCCCGGGTTATATGGGGT |
| | TTATATGGGAAAAGAGGTCTTTCCTTTTTTTTGGGGGGTATATTTTTTTT |
| | TTTAAAGGAGATCCAACCCCCGGGCTCTGGGGCTTTTAAAAAAAAAATTT |
| | TGGGGAGGTTCCCCGGGGGCCCTCCTCCTTAAAAAACCCCACCCCCCCGG |
| | GGTTTTTTTTCAAGGC |
| SEQ ID NO:22 | AGGN |
| | GGCCATATACAGTATCAGTGCTTTCCTGGTTATAAGCTCCATGGAAATTC |
| | ATCAAGAAGGTGCCTCTCCAATGGCTCCTGGAGCGGCAGCTCACCTTCCT |
| | GCCTGCCTTGCAGATGTTCCACACCAGTAATTGAATATGGAACTGTCAAT |
| | GGGACAGATTATGACTGTGGAAAGGCAGCCCGGATTCAGTGCTTCAAAGG |
| | CTTCACGCTCCTAGGACTGTCTGAAATCACCTGTGAAGCCGATGGCCAGT |
| | GGAGCTCTGTGTTCCCCCACTGTGAACACACTTCATGTGGTTCTCTTCCA |
| | ATGATACCAAATGCGGTCATCTCTTCTCGGAAGGGCCTGGCCATCCTGAA |
| | ACTCTGCAAAGAAATCCAACATGCGCTGGGCCTTTGTAAGTAAACCTGTA |
| | CCTTGAGTTACTTTTTTTATTAGGGGGAATAAATTGGGAATTCCTTGGAA |
| | AAAAATTATTAAATGGTGCATTTTAAAAAATCGCGGGTTTTCCTTTTAAA |
| | AATTTTTTAATTGGAGCTGCCTTACCTTAAAAAAAAATGAAATGTGGG |
| SEQ ID NO:23 | GAAATGGCCCCTTCCCCTTGAACC |
| | CTCTGTCATNNGTATAGGTNGNCNTCATGATAATTCAGTCGACATCGGNT |
| | CGCCATCTNANGATCTGNGGACATCTGCACGCGCNGAGGATACACAGTGC |
| | AGAACACATGTGGCGGGCACGCGTACTGAGATCCGCACACTAGCAGCCAA |
| | AAGCTCATTACCGCCCCGCATAGTTGCATAGTCATCTTAGCAGGAGCCGC |
| | CATCATGTAACATACATATCGTGAACGCTTACATTCACCGCATTGACACT |
| | TACATAAAAGATCCCAAAGAAAGGGAATTTCTCTTCAATGCCATTGAAAC |
| | GATGCCTTGTGTCAAGAAGAAGGCAGACTGGGCCTTGCGCTGGATTGNGG |
| | ACAAAGAGGCTACCTATGGTGAACGTGTTGTAGCCTTTGCTGCAGTGGAA |
| | GGCATTTTCTTTTCCGGTCTTTTGCGTCGATATTCTGGCTCAAGAAACGA |
| | GGACTGATGCCTGCCTCACATTTCTAATGGACTATATAGCGAGAATAGGG |
| | TTACACTGGGAATTTGCTTGCCTGAGGTCCAAACACCCGGCCCCAAACCT |
| | TCGGNGGGAAAGTAGGAGAATAATTTCATGCTGTCCGTATGAACACGGAT |
| | CCTAACTGAGGCTTGCCTGTAACGTAATTGGATGATTGCCCTCAATGAGC |
| | ATACTTTGGTTTTGGCAACAACATTCCTGAACCGGTTTACCAGTTTCAAA |
| | AAAAACCTTTGCTTTTGGAATTTCCCTGAGGAACTACTTTTGAAAAGAGG |
| | CCGTTAAAA |
| SEQ ID NO:24 | AGGGGNTCTTTTNNNNNGATGCCTCCTACCCTGATGATGGTGCGCAGATT |
| | AGTCGCNCGTGTGACGAGATCTGGACATATCGCACGGCGCATGGCGCCCA |
| | ACGCATAGCAGGACGCTCGCAGAAGCAGCATGAGCCCCGGCTCACATTCC |
| | CGCGCGAAGAACATGCGTAACCAACAGCGTGTCTGGACCACAGCCCTGTC |
| | ACCCTGACACTGAATCGCACGCAATGCTAGCTGCCCCTTTCCCGTCCTGG |
| | GCACCCCGAGTCTCCCCCGACCCCGGGTCCCAGGTATGCTCCCACCTCCA |
| | CCTGCCCCACTCACCACCTCTGCTAGTTCCAGACACCTCCACGCCCACCT |
| | GGTCCTCTCCCATCGCCCACAAAAGGGGGGGCACGAGGGACGAGCTTAGC |
| | TGAGCTGGGAGGAGCAGGGTGAGGGTGGGCGACCCAGGATTCCCCCTCCC |
| | CTTCCCAAATAAAGATGAGGGTACTAAAANAAAAAAAAAAAANAAAANNN |
| | NCCCCAGAAAGGTTTGGGTTTTTTCCCCAAGGGGTTGGGAAAGATTCCAA |
| | AAAAGGGGTGGCGTGGTGTGAAAANNNNNNAAAACCNNNNGNAATNGAAC |
| | CCTTTGTTATTCAAAAGCTTGTTGGGAAAAGGAAAACCCCCCCCCTTTGA |
| | ACTAACAATTTTTAAAATTGAACTGTTACTAAACAGAAAAAAAAGTTTTT |
| | GGTTTTTTTTGATCTGACTGTAATGAAAANNNNATTTTTTCCTAGGGTTT |
| | TAAAGAGTAATACTTTTTGTAAAACTCTTTGGAAGTGGGCCTTTGGAAAG |
| | GAAAAAATTGTTTTNTAGGGAAACTATTTAAAG |
| SEQ ID NO:25 | AAGGACTACCCAGTGGTGTCTATCGAAGAT |
| | CCCTTTGACCAGGATGACTGGGGAGCTTGGCGAGAAGTTCACAGCCAGTG |
| | CAGGAATCCAGGTAGTGGGGGATGATCTCACAGTGACCAACCCAAAGAGG |
| | ATCGCCAAGGCCGTGAACGAGAAGTCCTGCAACTGCCTCCTGCTCAAAGT |
| | CAACCAGATTGGCTCCGTGACCGAGTCTCTTCAGGCGTGCAAGCTGGCCC |
| | AGGCCAATGGTTGGGGCGTCATGGTGTCTCATCGTTCGGGGGAGACTGAA |
| | GATACCTTCATCGCTGACCTGGTTGTGGGGCTGTGCACTGTGCAGATCAA |
| | GACTGGTGCCCCTTGCCGATCTGAGCGCTTGGCCAAGTACAACCAGCTCC |
| | TCAGAATTGAAGAGGAGCTGGGCAGCAAGGCTAAGTTTGCCGGCAGGAAC |
| | TTCAGAAACCCCTTGCCAAGTAAGCTGTGGGCAGGCAAGCCCTTCGGTCA |
| | CCTGTTGTCTACACAGANCCCTTCCCTCGTGTCAGCTCAGGCAGCTCGAG |
| | GCCNNCGACCAACACTTGCAGGGGTCCNTTGCTAGTAGCGCCCCACCCGC |
| | GTGGAGTTCGTACCGCTTCTTTAGACTTCNTACAGAAGCCAAGCTTCCTT |
| | GGAGCCCTG |
| SEQ ID NO:26 | GGATGCCTCCTACCTCTGATGATGTGCCAT |
| | AATTAGTCACCTGTCACGGATTCGAATCGAGCGCGGACGAGTCGACCATG |
| | CTGTGCGCGCGAGGCGACCAGCGGGCGCTCTAACAGCCGCCTGATCGCGG |
| | ACCTGTTGAGCGCCGACTAAGACTAGACGTTATTGACCACTCACGTGAAC |
| | CTACTAGCCCACAGGCGGTTTTGTGAGCTGCTTCCCCAGGAGCAGCGGAG |
| | CGTGGAGTCGTCACTTCGGGCACAAGTGCCCTTCGAGCAGATTCTCAGCC |
| | TTCCAGAGCTCAAGGCCAACCCCTTCAAGGAGCGAATCTGCAGGGTCTTC |
| | TCCACATCCCCAGCCAAAGACAGCCTTAGCTTTGAGGACTTCCTGGATCT |
| | CCTCAGTGTGTTCAGTGACACAGCCACGCCAGACATCAAGTCCCATTATG |
| | CCTTCCGCATCTTTGACTTTGATGATGACGGAACCTTGAACAGAGAAGAC |
| | CTGAGCCGGCTGGTGAACTGCCTCACGGGAGAGGGCGAGGACACACGGCT |
| | TANTGCGTCTGAGATGAAGCAACTCATCGACAACATTCTGGAGGAGTCTG |
| | ACATTGACAGGATGGACCATCAACTCTCTGAGTNCAGCACGTNATCTCCC |
| | GTCTTCAGACTTTGCAAGTTCTTTAGAATGCCTGTGACAGAACCCCAGCT |
| | GGGTCTGGACCTTGTCAAAACCTTTACTGTGACTTTGGCAAGTAAACTTG |
| | TTGCAATGCGGCCACTTGGCAACTGACTGG |
| SEQ ID NO:27 | TGTGGACCTCGTCGATGAACAGCACTCC |
| | TTCCTCAACCGGGCCCTGGAGAGTGACATGGCGCCTGTCCTGATCATGGC |
| | CACCAACCGTGGCATCACGCGAATCCGGGGCACCAGCTACCAGAGCCCTC |
| | ACGGCATCCCCATAGACCTGCTGGACCGGCTGCTTATCGTCTCCACCACC |
| | CCCTACAGCGAGAAAGACACGAAGCAGATCCTCCGCATCCGGTGCGAGGA |
| | AGAAGATGTGGAGATGAGTGAGGACGCCTACACGGTGCTGACCCGCATCG |
| | GGCTGGAGACGTCACTGCGCTACGCCATCCAGCTCATCACAGCTGCCAGC |
| | TTGGTGTGCCGGAAACGCAAGGGTACAGAAGTGCAGGTGGATGACATCAA |
| | GCGGGTCTACTCACTCTTCCTGGACGAGTCCCGCTCCACGCAGTACATGA |
| | AGGAGTACCAGGACGCCTTCCTCTTCAACGAACTCAAAGGCGAGACCATG |
| | GACACCTCCTGAGTTGGATGTCATCCNCCGACCCCACCCTGTTTTCCACC |
| | AGAGTTCTGACACTGTGACTCTGTATAAAATGGGTGGGAAGCTGCACCCA |
| | CCCTGTGTATGTGTGGTTGCCCTGAGCCCNCNGAATGCCANAAAATAAAA |
| | AATAATTCCTTAGAAG |
| SEQ ID NO:28 | AAGACGCAGCTGACATCAATGCTCATGA |
| | TGAACCTGGAATCCAGGCCTGTGATCTTCGAGGATGTGGGGAGGCAGGTG |
| | CTGGCCACTCGCTCCAGAAAGCTGCCGCACGAGCTGTGCACGCTCATCCG |
| | CAACGTGAAGCCGGAAGATGTGAAGAGAGTCGCTTCTAAGATGCTCCGAG |
| | GGAAGCCGGCAGTGGCCGCCCTGGGTGACCTGACTGACCTGCCCACGTAT |
| | GAGCACATCCAGACCGCCCTGTCGAGTAAGGACGGGCGCCTGCCCAGGAC |
| | GTACCGGCTCTTCCGGTAGAACCGCTCCCCGGCCTGACAGACCCAGGGAG |
| | CTGCAGCTGGAGCCCGTTCCCGTGCGTGTTAGTTTGTACACGAATTTAGT |
| | CTAAAAAGCTGTCTGGTTGTATAAACGGTGCAAACAATGTCGCCACAGCA |
| | CCCACGCGGATTGCATTCTTTTGGAACTCAATGTGCCGATCAGTGGAGTC |
| | AGTATCGAGCCTGACCACCGCAAGCCAGGAAGCANGTGAAGTGCCCAGCG |
| | CTGGAGTGCATCGTGCCACGAGGAGGGCGGTCGGTGCTTCCCTTCTCGAG |
| | CTGTGGGCACATAGCGCCCCGCAGGTTCCTTGGATGTAGCCCTGATCTAG |
| | GTAGCACC |
| SEQ ID NO:29 | CTCTTTTNTTTATCCTCCTACTTGATGATGTGCGAAA |
| | TCAGTACCGCTGACGAACTGGGAACTGAGCGGCGGATACTGGAGTGGCAT |
| | CGACAAGTCGAATCGAGGTCGCACCAAGCGGCGACAGCTGATAACCATCA |
| | CGAACAGCCTTGCATCATTGAGCACCGCATCACTGCCAACAGTTGTAGGC |
| | ACGACTAACATCCACTCGCAAGGGCAGAAGGTTGAAGAACAGGAGCCTGA |
| | ACTGACATCAACTCCCAATTTCGTGGTTGAAGTTATAAAGAATGATGATG |
| | GCAAGAAGGCCCTTGTGTTGGACTGTCATTATCCAGAGGATGAGGTTGGA |
| | CAAGAAGACGAGGCTGAGAGTGACATCTTCTCTATCAGGGAAGTTAGCTT |
| | TCAGTCCACTGGCGAGTCTGAATGGAGGATACTAATTATACACTCAACAC |
| | AGANTCCTTGGACTGGCCCTTATATGACCACCCTATGAATTTCCTTGCCG |
| | ACCGAGGGGGTGACAACACTTTTGCCAGATAACCGGTGGAACTCAGCCCA |
| | AGCCTTGAGCAACAGGGAGTCCATTACTTTTCTTGGAGAACCTTAGGAAA |
| | TTTTGTCAAGAGAGCCCTTTAAACCCCCACCAATGCCTGAAAAGCCCTTA |
| | GTTTTCAATGGGCAGGGCCTTTGGCCCCAGGGGAACAAAAAACCCTCACC |
| | CTTTAAAAGCTTTAACAACTGGGCCCTTTTGGAAAAGGGGAGTTTTCAAC |
| | CCCCCAAAATCCCAAAAGGGGGGGAAAAAAAACCCCCCCAATTTTAAAAA |
| | ATTTTTTTGGGTTTGGGGGGGGGGCCCCCAATATTAAAATAAAAAAATTT |
| | TTTTTTTCTGTTGACACAAAAAA |

nucleotide in upper case: high quality
nucleotide in lower case: low quality
*: pad or gap to maximize alignment
N, n: repeat-masked or uncertain
X, x: cross-matched

TABLE 3
BLAST alignment data for interactors

| | | | Percent | | | | |
|---|---|---|---|---|---|---|---|
| Interactor | Hit ID | Hit Annotation | ID | Overlap | Score | P Value | UG Cluster |
| Contig4097 | X13293 | Human mRNA for B-myb gene | 99 | 499 | 981 | 0 | Hs.179718 |
| | | /cds = (127, 2229)/gb = X13293 | | | | | |
| | | /gi = 29471/ug = Hs.179718/len = 2627 | | | | | |
| Contig4098 | U01038 | Human pLK mRNA, complete cds | 98 | 954 | 1792 | 0 | Hs.77597 |
| | | /cds = (63, 1874)/gb = U01038 | | | | | |
| | | /gi = 393016/ug = Hs.77597/len = 2178 | | | | | |
| Contig4099 | X75315 | H. sapiens seb4B mRNA/cds = (0, 693) | 97 | 281 | 496 | 0 | Hs.247500 |
| | | /gb = X75315/gi = 407420 | | | | | |
| | | /ug = Hs.247500/len = 1438 | | | | | |
| Contig4100 | AF086904 | Homo sapiens protein kinase Chk2 | 95 | 418 | 646 | 0 | Hs.146329 |
| | | (CHK2) mRNA, complete cds | | | | | |
| | | /cds = (0, 1631)/gb = AF086904 | | | | | |
| | | /gi = 3982839/ug = Hs.146329/len = 1735 | | | | | |
| Contig4101 | no hit | unknown sequence | | | | | |
| Contig4103 | S57501 | protein phosphatase type 1 catalytic | 98 | 635 | 1180 | 0 | Hs.183994 |
| | | subunit [human, mRNA, 1400 nt] | | | | | |
| | | /cds = (11, 1036)/gb = S57501 | | | | | |
| | | /gi = 298963/ug = Hs.183994/len = 1388 | | | | | |
| Contig4104 | H57957 | yr12h06.s1 Homo sapiens cDNA, 3′ | 89 | 305 | 357 | 9E-98 | Hs.230106 |
| | | end/clone = IMAGE:205115 | | | | | |
| | | /clone_end = 3′/gb = H57957 | | | | | |
| | | /gi = 1010789/ug = Hs.230106/len = 390 | | | | | |
| Contig4105 | AL050141 | Homo sapiens mRNA; cDNA | 99 | 332 | 642 | 0 | Hs.227834 |
| | | DKFZp586O031 (from clone | | | | | |
| | | DKFZp586O031)/cds = UNKNOWN | | | | | |
| | | /gb = AL050141/gi = 4884352 | | | | | |
| | | /ug = Hs.227834/len = 2353 | | | | | |
| Contig4707 | U46025 | Human translation initiation factor eIF- | 100 | 722 | 1431 | 0 | Hs.4835 |
| | | 3 p110 subunit gene, complete cds | | | | | |
| | | /cds = (0, 2741)/gb = U46025 | | | | | |
| | | /gi = 1718196/ug = Hs.4835/len = 2742 | | | | | |
| Contig5000 | DSR2_HUMAN | DOWN SYNDROME CRITICAL | 94 | 168 | 328 | 1E-89 | none |
| | | REGION PROTEIN 2 (LEUCINE | | | | | |
| | | RICH PROTEIN C21-LRP). | | | | | |
| Singlet6481 | AL117589 | Homo sapiens mRNA; cDNA | 95 | 228 | 361 | 6E-99 | Hs.134970 |
| | | DKFZp434N178 (from clone | | | | | |
| | | DKFZp434N178)/cds = (0, 808) | | | | | |
| | | /gb = AL117589/gi = 5912152 | | | | | |
| | | /ug = Hs.134970/len = 1907 | | | | | |
| Singlet6482 | AJ132583 | Homo sapiens mRNA for puromycin | 99 | 364 | 706 | 0 | Hs.132243 |
| | | sensitive aminopeptidase, partial | | | | | |
| | | /cds = (85, 2712)/gb = AJ132583 | | | | | |
| | | /gi = 4210725/ug = Hs.132243/len = 4049 | | | | | |
| Singlet6484 | D42044 | Human mRNA for KIAA0090 gene, | 97 | 505 | 805 | 0 | Hs.154797 |
| | | partial cds/cds = (0, 2718)/gb = D42044 | | | | | |
| | | /gi = 577300/ug = Hs.154797/len = 5726 | | | | | |
| Singlet6487 | AF034799 | Homo sapiens liprin-alpha2 mRNA, | 96 | 642 | 1072 | 0 | Hs.30881 |
| | | complete cds/cds = (169, 3942) | | | | | |
| | | /gb = AF034799/gi = 3309532 | | | | | |
| | | /ug = Hs.30881/len = 4060 | | | | | |
| Singlet6488 | AB028998 | Homo sapiens mRNA for KIAA1075 | 99 | 345 | 662 | 0 | Hs.6147 |
| | | protein, partial cds/cds = (0, 4202) | | | | | |
| | | /gb = AB028998/gi = 5689486 | | | | | |
| | | /ug = Hs.6147/len = 4692 | | | | | |
| Singlet6489 | X66276 | H. sapiens mRNA for skeletal muscle | 96 | 493 | 821 | 0 | Hs.169849 |
| | | C-protein/cds = (96, 3512)/gb = X66276 | | | | | |
| | | /gi = 36500/ug = Hs.169849/len = 3833 | | | | | |
| Singlet6491 | no hit | unknown sequence | | | | | |
| Singlet6492 | no hit | unknown sequence | | | | | |
| Singlet6497 | G19371 | human STS SHGC-17415. | 96 | 199 | 325 | 1E-86 | none |
| Singlet6498 | AL079279 | Homo sapiens mRNA full length insert | 97 | 321 | 565 | 0 | Hs.8963 |
| | | cDNA clone EUROIMAGE 248114 | | | | | |
| | | /cds = UNKNOWN/gb = AL079279 | | | | | |
| | | /gi = 5102585/ug = Hs.8963/len = 2428 | | | | | |
| Contig4563 | X59618 | H. sapiens RR2 mRNA for small | 97 | 1492 | 2623 | 0 | Hs.75319 |
| | | subunit ribonucleotide reductase | | | | | |
| | | /cds = (194, 1363)/gb = X59618 | | | | | |
| | | /gi = 36154/ug = Hs.75319/len = 2475 | | | | | |
| Contig5071 | O55215 | RIBOSOMAL PROTEIN S2. | 99 | 240 | 486 | 0 | none |
| Contig5085 | ENOA_MOUSE | ALPHA ENOLASE (EC 4.2.1.11)(2- | 94 | 315 | 612 | 0 | none |
| | | PHOSPHO-D-GLYCERATE HYDRO- | | | | | |
| | | LYASE)(NON-NEURAL | | | | | |
| | | ENOLASE)(NNE). | | | | | |
| Contig5087 | CIB_HUMAN | SNK INTERACTING PROTEIN 2-28 | 90 | 184 | 326 | 1E-88 | none |
| | | (SIP2-28)(CALCIUM AND | | | | | |
| | | INTEGRIN-BINDING PROTEIN CIB) | | | | | |
| | | (KIP). | | | | | |
| Contig5185 | gnl\|UG\|Hs#S5565 | gnl\|UG\|Hs#S5565 Human mRNA for | 100 | 320 | 634 | 0 | none |
| | | U1 small nuclear RNP-specific C . . . | | | | | |
| Singlet6483 | Q16704 | ENOLASE (EC 4.2.1.11)(2- | 99 | 315 | 637 | 0 | none |
| | | PHOSPHOGLYCERATE | | | | | |
| | | DEHYDRATASE)(2-PHOSPHO-D- | | | | | |
| | | GLYCERATE HYDRO-LYASE). | | | | | |
| Singlet6499 | ENOA_HUMAN | ALPHA ENOLASE (EC 4.2.1.11)(2- | 100 | 315 | 640 | 0 | none |
| | | PHOSPHO-D-GLYCERATE HYDRO- | | | | | |
| | | LYASE)(NON-NEURAL | | | | | |
| | | ENOLASE)(NNE) | | | | | |
| | | (PHOSPHOPYRUVATE | | | | | |
| | | HYDRATASE). | | | | | |

That which is claimed is:

1. A method of screening a test compound for the ability to inhibit binding of Polo-like kinase 3 to a protein selected from the group consisting of
a) proteins listed in Table 1 herein;
b) proteins comprising an amino acid sequence encoded by a nucleotide sequence selected from SEQ ID NOS:3-29;
c) a fragment of a protein of (a) or (b) above, said fragment comprising an Polo-like kinase 3 binding site;
comprising selecting one of said proteins and detecting whether said test compound inhibits binding of said selected protein to Polo-like kinase 3, compared to that which would occur in the absence of said test compound.

2. A method of screening a test compound for the ability to bind Polo-like kinase 3 at the binding site for a binding protein selected from the group consisting of the proteins provided in Table 1, comprising:
(a) selecting a binding protein;
(b) contacting a test compound to Polo-like kinase 3, or to a portion of Polo-like kinase 3 sufficient to bind said selected binding protein;
(c) contacting said selected binding protein to said Polo-like kinase 3 or portion thereof; and
(d) detecting whether said test compound inhibits binding of said selected protein to said Polo-like kinase 3, compared to that which would occur in the absence of said test compound.

3. A method according to claim 2 wherein said contacting step is carried out in vitro.

4. A method of identifying a compound which interferes with the binding of Polo-like kinase 3 to a pre-selected protein, said method comprising the steps of:
forming a mixture by combining a labeled first protein with a second protein, wherein one protein is Polo-like kinase 3 or a binding fragment thereof and the other protein is selected from the group consisting of proteins in Table 1 herein;
contacting a test compound to the mixture; and
determining the quantity of the first protein which is bound to the second protein before and after said adding step, wherein a decrease in the quantity of the first protein which is bound to the second protein after the adding step indicates that the test compound interferes with the binding of Polo-like kinase 3 to said selected protein.

5. A method according to claim 3, wherein said contacting step is carried out in vitro.

6. A method of screening allelic variants of Polo-like kinase 3 for altered protein binding capability, comprising:
a) obtaining an allelic variant of Polo-like kinase 3;
b) selecting an interactor protein from the group consisting of proteins of Table 1 herein;
c) comparing the binding of said allelic variant of Polo-like kinase 3 and said interactor protein to that of a different allelic variant of Polo-like kinase 3.

7. A method of inhibiting a physiologic pathway where said pathway includes the step of Polo-like kinase 3 binding to a protein selected from the proteins of Table 1 herein, comprising inhibiting the binding of Polo-like kinase 3 to said selected protein.


SEQUENCE LISTING (29 sequences)

SEQ ID NO: 1 - DNA, 2169 bp, homo sapiens
  CDS (37)..(1860)
ccgcctccgagtgccttgcgcggacctgagctggagatgctggccgggctaccgacgtcagaccccgggcgcctcatcacggacccgcgcagcggccgcacctacctcaaaggccgcttgttgggcaaggggggcttcgcccgctgctacgaggccactgacacagagactggcagcgcctacgctgtcaaagtcatcccgcagagccgcgtcgccaagccgcatcagcgcgagaagatcctaaatgagattgagctgcaccgagacctgcagcaccgccacatcgtgcgtttttcgcaccactttgaggacgctgacaacatctacattttcttggagctctgcagccgaaagtccctggcccacatctggaaggcccggcacaccctgttggagccagaagtgcgctactacctgcggcagatcctttctggcctcaagtacttgcaccagcgcggcatcttgcaccgggacctcaagttgggaaattttttcatcactgagaacatggaactgaaggtgggggattttgggctggcagcccggttggagcctccggagcagaggaagaagaccatctgtggcacccccaactatgtggctccagaagtgctgctgagacagggccacggccctgaagcggatgtatggtcactgggctgtgtcatgtacacgctgctctgcgggagccctccctttgagacggctgacctgaaggagacgtaccgctgcatcaagcaggttcactacacgctgcctgccagcctctcactgcctgcccggcagctcctggccgccatccttcgggcctcaccccgagaccgcccctctattgaccagatcctgcgccatgacttctttaccaagggctacacccccgatcgactccctatcagcagctgcgtgacagtcccagacctgacaccccccaacccagctaggagtctgtttgccaaagttaccaagagcctctttggcagaaagaagaagagtaagaatcatgcccaggagagggatgaggtctccggtttggtgagcggcctcatgcgcacatccgttggccatcaggatgccaggccagaggctccagcagcttctggcccagcccctgtcagcctggtagagacagcacctgaagacagctcaccccgtgggacactggcaagcagtggagatggatttgaagaaggtctgactgtggccacagtagtggagtcagccctttgtgctctgagaaattgtatagctttcatgcccccagcggaacagaacccggcccccctggcccagccagagcctctggtgtgggtcagcaagtgggttgactactccaataagttcggctttgggtatcaactgtccagccgccgtgtggctgtgctcttcaacgatggcacacatatggccctgtcggccaacagaaagactgtgcactacaatcccaccagcacaaagcacttctccttctccgtgggtgctgtgccccgggccctgcagcctcagctgggtatcctgcggtacttcgcctcctacatggagcagcacctcatgaagggtggagatctgcccagtgtggaagaggtagaggtacctgctccgcccttgctgctgcagtgggtcaagacggatcaggctctcctcatgctgtttagtgatggcactgtccaggtgaacttctacggggaccacaccaagctgattctcagtggctgggagcccctccttgtgacttttgtggcccgaaatcgtagtgcttgtacttacctcgcttcccaccttcggcagctgggctgctctccagacctgcggcagcgactccgctatgctctgcgcctgctccgggaccgcagcccagcttaggacccaagccctgaaggcctgaggcctgtgcctgtcaggctctggcccttgcctttgtggccttcccccttcctttggtgcctcactgggggctttgggccgaatcccccagggaatcagggaccagctttactggagttgggggcggcttgtcttcgctggctcctaccccatctccaagataagcctgagccttagctcccagctagggggcgttatttatggaccacttttatttattgtcagacacttatttattgggatgtgagccccagggggcctcctcctaggataataaacaattttgca

SEQ ID NO: 2 - protein, 607 aa, homo sapiens
MLAGLPTSDPGRLITDPRSGRTYLKGRLLGKGGFARCYEATDTETGSAYAVKVIPQSRVAKPHQREKILNEIELHRDLQHRHIVRFSHHFEDADNIYIFLELCSRKSLAHIWKARHTLLEPEVRYYLRQILSGLKYLHQRGILHRDLKLGNFFITENMELKVGDFGLAARLEPPEQRKKTICGTPNYVAPEVLLRQGHGPEADVWSLGCVMYTLLCGSPPFETADLKETYRCIKQVHYTLPASLSLPARQLLAAILRASPRDRPSIDQILRHDFFTKGYTPDRLPISSCVTVPDLTPPNPARSLFAKVTKSLFGRKKKSKNHAQERDEVSGLVSGLMRTSVGHQDARPEAPAASGPAPVSLVETAPEDSSPRGTLASSGDGFEEGLTVATVVESALCALRNCIAFMPPAEQNPAPLAQPEPLVWVSKWVDYSNKFGFGYQLSSRRVAVLFNDGTHMALSANRKTVHYNPTSTKHFSFSVGAVPRALQPQLGILRYFASYMEQHLMKGGDLPSVEEVEVPAPPLLLQWVKTDQALLMLFSDGTVQVNFYGDHTKLILSGWEPLLVTFVARNRSACTYLASHLRQLGCSPDLRQRLRYALRLLRDRSPA

SEQ ID NO: 3 - DNA, 651 bp, Artificial - cDNA prey sequence
  misc_feature (451)..(509): N=any nucleotide
attggagctggagagcccctcgctgacatccaccccagtgtgcagccagaaggtggtgggcgaccacaccactgcaccgggacaagacacccctgcaccagaaacatgctgcgtttgtaaccccagatcagaagtactccatggacaacactccccacacgccaaccccgttcaagaacgccctggagaagtacggacccctgaagcccctgccacagaccccgcacctggaggaggacttgaaggaggtgctgcgttctgaggctggcatcgaactcatcatcgaggacgacatcaggcccgagaagcagaagaggaagcctgggctgcggcggagccccatcaagaaagtccggaagtctctggctcttgacattgtggatgaggatatgaagctgatgatgtccacatctcccctccactcccctgcttaataaactctaaaaatccngnngngaaaaaggnaannnnngaannncagncnaagggagcaaggaaaagaaaaannngccgcggggggtgttttcctttttttgcacgggtagggggtcatcccccaaaatgaggttgggttggaaaaaaaaatcctgcttaaaaccacaagaaacttgtttcacttattaggaaggaaaagattaattaaaatggccg

SEQ ID NO: 4 - DNA, 637 bp, Artificial -cDNA prey sequence
  misc_feature (500)..(640): n=any nucleotide
gaggttcgagagacaggtgaggtggtcgactgccacctcagtgacatgctgcagcagcggcacagtgtcaatgcctccaagccctcggagcgtgggctggtcaggcaagaggaggctgaggatcctgcctgcatccccatcttctgggtcagcaagtgggtggactattcggacaagtacggccttgggtatcagctctgtgataacagcgtgggggtgctcttcaatgactcaacacgcctcatcctctacaatgatggtgacagcctgcagtacatagagcgtgacggcactgagtcctacctcaccgtgagttcccatcccaactccttgatgaagaagatcaccctccttaaatatttccgcaattacatgagcgagcacttgctgaaggcaggtgccaacatcacgccgcgcgaaggtgatgagctcgcccggctgccctacctacggacctggttccgcacccgcagcgccatcatcctgcacctcagcaacggcagcgtgcagatcaacttcttccatgatcacaccaagctcatcttgtgcccactgatggcagccgtgacctacatcgacgagaagcgggacttnccgcacataccgnctgagtctnctggaggagtacggctgctga

SEQ ID NO: 5 - DNA, 559 bp, Artificial -cDNA prey sequence
  misc_feature (332)..(332): N=any nucleotide
ttatgctccagcttgtaccgagcttagacatactagtcacggctgcgcagtgtggtgggaattcgaatgcttgggggcgtggaatgtggtagaagaagcagactgaatttactgacagacaggttagcattaaaagattcacaggatatacgctgcaacttcagcgctacgactggaaaggggcctttggccggcggcccctgttaccggcggcccctgtgcgcctgggagctcctccgggcttgaggaagccgcccacgtgccctgatggagaaaatgggactccaacaggaggccgtgtcctcacacctcagactgcgctcacagctcgngaggatcaagttacaataaacagtccattaacttcttgctttcaggtttccctggagtcaggcatctctgcacagtccaggcagcccagggctgcagagggctgtacacccgccacatcacagtgggacacagctgagactgagtggaagcagaaagtcagaagctcatggcagactgatgcctatagtagatcatccatgcgcgcagtctaagcgctatgttactt

SEQ ID NO: 6 - DNA, 550 bp, Artificial - cDNA prey sequence
ctctcactccagctctgggacactgagctccttagagacagtgtccactcaggaactctattctattcctgaggaccaagaacctgaggaccaagaacctgaggagcctacccctgccccctgggctcgattatgggcccttcaggatggatttgccaatcttgaatgtgtgaatgacaactaccggtttgggagggacaaaagctgtgaatattgctttgatgaaccactgctgaaaagaacagataaataccgaacatacagcaagaaacactttcggattttcagggaagtgggtctaaaaacttttacattggataccttagaaaatacagtggcaatggaaacctttgtaattccagaacttgtagggaaaggaaaacccccctcttttgaataaccattcttaaattgcccttgtacttaaccggaaataaaggttttggcttttttgaaccgaccgggaaaaaacaaaccagtttatctctagggctttaaggaatgaatccttttgtcaaaaaccttttgaatgggcccctttgaaaggaaa

SEQ ID NO: 7 - DNA, 405 bp, Artificial - cDNA prey sequence
aattgacgactgctgctggcacatggagcccctctcgccaattcccattgaccactggaacctggagcggaccggccccctgagcaccagcagccccagccgcaggatgaacgaggccgccgacagccgtgactgtcgctccccgggactcctggacaccacccccatccgaggaagctgcactacccagaggaaattgcaagagaagtcctcgggcgcgggctccctggggaatagcaggccgagctttctgaattcggctctgtgggacgtttgggacggggaagagcagaggcctccagagacccctcctccggcccagatgccaagcgctggtggagctcagaagcccgaagggttagagacacccaaaggtgctaatcggaagaagaacttgccccgaat

SEQ ID NO: 8 - DNA, 634 bp, Artificial -cDNA prey sequence
ctctttctgggggactatgtggacaggggcaagcagtccttggagaccatctggctgctgctggcctataagatcaagtaccccgagaacttcttcctgctccgtgggaaccacgagtgtgccagcatcaaccgcatctatggtttctacgatgagtgcaagagacgctacaacatcaaactgtggaaaaccttcactgactgcttcaactgcctgcccatcgcggccatagtggacgaaaagatcttctgctgccacggaggcctgtccccggacctgcagtctatggagcagattcggcggatcatgcggcccacagatgtgcctgaccagggcctgctgtgtgacctgctgtggtctgaccctgacaaggacgtgcagggctgtggcgagaacgaccgtggcgtctcttttacctttggagccgaggtggtggccaagttcctccacaagcacgacttggacctcatctgccgagcacaccaggtggtagaagacggctacgagttctttgccaagcggcagctggtgacacttttctcagctcccaactactgtggcaaggttgacaaatgctgcggccatgatgagtgtggacgagaccctcatgtgctcttttcagatcctcaagccc

SEQ ID NO: 9 - DNA, 587 bp, Artificial - cDNA prey sequence
  misc_feature (528)..(551): n=any nucleotide
cctggctcctactccaggtcccccgcggggtcccagcagcaattcggctactccccagggcagcagcagacccacccccagggttctccaaggacatctacaccatttggatcagggcgtgttagagaaaaaagaatgtctaatgagttggaaaattatttcaagccttcaatgcttgaagatccttgggctggcctagaaccagtatctgtagtggatataagccaacaatacagcaatactcaaacattcacaggcaaaaaaggaagatacttttgttaacatttctgaaattcaactggaagcttcatgtgtcaggaacatcttggacaaaactttaagttgtgttgatataaatttacccaaagatgatgactttgattggataattagtaaggtctttttgttatttttcatcgtatcaggtattgttgatattagagaaaaaagtaggataacttgcaacatttagctctggaagtacctaccacaatttagagatttaccgtttccatatatttaacattnctggtacantatgggacattgnnctttaatgttttttcaatgttttaaaaataaacatt

SEQ ID NO: 10 - DNA, 646 bp, Artificial -cDNA prey sequence
  misc_feature (591)..(629): n = any nucleotide
agagagagagagagaggagaaagtgagctcagcgagttggccgggtgacacactgatgagggggtcaaaggacactctgagttagtgccctcggcacacacagcgaacagtgatcatgaaaagagtgggctcaataattttccataaacttgctcaagattccatgcagttgccatacagtctttgaggtatggtcaacctatagtaagttagtaaatgttaaggggaggaagaaatggaaacctaaacatctactgcaatgaaaaccaacagccatgtcagtaggagtaattcaaccttcgttgaacacatgaaattgaacacactcttgttttccctggacctggcatctccaggtgtcaacacagaattaagcatccataattgctcaaagttacctggcgcatgatgggtcttggtcttcttacacttcttggtacttttcaatttcatccatgtcaacagccaagccaacacactgttgctccaatatgtaaaaggcacttctgtagggctggcatgagtcagtcagttcaagacaacctgaaggagttgaataacatctatccagtgagttctgcaagacttgangctctttctcatccagcagctctctgctgagcctgaanaagtgagaaaaagaaaa

SEQ ID NO: 11 - DNA, 859 bp, Artificial - cDNA prey sequence
  misc_feature (1)..(776): n = any nucleotide
nttttttnnnnnaggcctcctagctctgatgatgcgcgatgatcagtcgcttctcacgagattcggaacgaggcggagaagttggagcaaggctggccgagaacagatgaacgggagcccgactacatgctagggccacctagcggcgttacttccgagaccacatggacggctaccgcaaaaattagaccttacatgtgccgcggtggctaccgccagcagccgcctcagaccggcctactgagctctcccacctctgcatcccgcctgggccatccaaccttgaagtcctaaaccacacctcagtcactaaaggtctgtttaaagttaaaaaaaaaaaaaaaaaaaaaaaaaccccgggggggttggtgctttttccccaagggtttgggcaaaccccccaaaaagggtgcgggtttttaannnnnntnncccncanccnnnnnattttgcttttattcaacccctgggttgaaaagaacataataaaatacccganccttccccgcaaagaaacaccttttcgggatttttcaggggaagggggggcccctaaaaaaacctctttaacatttgccttcccctngaaaaaatcccccgggggcccatttgaaaccccctttttaaaaacccacaaccctttgtnagggaaaaaggaaaaaccccccccccccttttgaataaacaattttcttgaaatatgcccccggccccctaacgcaagaaaaaaaaggtttttggcctttttttgggaccccccctggggaaaaaaccanccccttttttctccccaggcctttaaagagaagaaaaaccttttttgtaaaaaactttttggaaaggggccccctggagagaa

SEQ ID NO: 12 - DNA, 596 bp, Artificial -cDNA prey sequence
  misc_feature (583)..(596): n=any nucleotide
gcgcaagccggcgtgcggtcccgcggcgctgcagttgtgtccagccggtcacggggcgggtatggcggccacgttcttcggagaggtggtgaaggcgccgtgccgagctgggactcgcccgaggacagggaggtgcgtctgcagctggcgcggaagagggaagtgcggctccttcgaagacaaacaaaaacatctttggaagtttctttgctagaaaaatatccgtgctccaagtttataattgctataggaaataatgcagtagcatttctgtcatcatttgttatgaattcaggagtctgggaggaagttggttgtgctaaactctggaatgaatggtgtagaacaacagacactacacatctgtcctccacagaggctttttgtgtgttttatcatctaaaatccaatccctcggtttttctctgtcagtgcagttgctatgttgcagaagatcaacagtatcagtggctggaaaaggtttttggctcttgtccaaggaagaacatgcagataactattctcacatgtcgacatgttaccgattataaaacctcagaatccaccggcagccttccttctnctttnctgagagn

SEQ ID NO: 13 - DNA, 594 bp, Artificial -  cDNA prey sequence
  misc_feature (402)..(563): n = any nucleotide
gggttgtgggggatctgtgtggggttctcaacgcagatccatcctggggtctcccgggcggggatggctgacctcgagtcccctcccttcccgagaacccgctctgtcccgagggcagctaacaagggctgagccccaggtacaggttgcctcttccacggcaggaatttttaccaaaaccacaagcaaaaaacaaaacagaccaccacgaccaacaacaaagatggggggtagggttttgtaaaggttctgttaggttcatatttttatatcattttgcccataaatgcggaatttgccgtgggaatttgaagacaaatgatctatgtttttatggttctctagggaaggtgttctgagggccgtgctctctccagctgtgggaggcctgctccctctggngggcaccctgngcagtgtgtggggcctttggaggcgctcttgccaatgcnacgagtgtgagcctgcagcgttgnacgtcccgacgaagctatacttctgagatcggctagatagacgctgaccttgacaatgttgatacatctgctcagcttattgtgatnagatgctcatggtaaaaaaaaaaataaaaac

SEQ ID NO: 14 - DNA, 652 bp, artificial - cDNA prey sequence
  misc_feature (9)..(579): n=any nucleotide
cggagtgtnnnntttgatgaccgaccccttggccaaaaaaaaacaaaaagcaaaaaacaaaaacctaccctgttctgggttttttcctccctttagttccacccccaacccccattccctggtgtccttcttagagatgaagaaataatacggaaacatctttcatagccacattaaataagagaaactgatatacattatttttttctttttaaagatgacttataagaaccctgaaatttatataggtgagacaatagaaataaaaagatcttcagccaggcctttctgaaggagttattctgctaaaaatggtcttagttgtctgaaaagccagctcttgaacctcttcacaacagtatcaacactggcttctcccggttcattttatgcgtgcgagaagtcagtggtaactgctgcagggcttaatacattagtggtaactggtttaaaaaacaaagactgtaagcctgtgtgtgccactgtttgcttcaacagtatatcctactaataagcctcactatttaatccaatgagttttaaatctaaatctcattcccttcttctttccctaccttnttttcttttgttcttaaaaaaatattttgtgtatttacagaaattcattattgggtggcttaacggattccag

SEQ ID NO: 15 - DNA, 789 bp, artificial - cDNA prey sequence
  misc_feature (13)..(16): n = any nucleotide
cggggctcttttnnnngatgcctcctagcctgatgatgtgcgaaatcagtcgccggtgacgaactggaaactgacgcgcgaacgagtctgaccgtgcgtggagcgtttaagaggacacttgagcaatgcataagccagcgcgtaatagcttgctggaccggggccagatgatgtaggtagttcagcaacgctatcatttaccgacctccatcagtgccatggaggccaccatcaccgaacggggcatcaccagccgacacctgctgattggactaccttctggagcaattctttcccttcctaaggctttgctggatccccgccgccccgagatcccaacagaacaaagcagagaggagaacttaatcccgtattctccagatgtacagatacacgcagagcgattcatcaactataaccagacagtttctcgaatgcgaggtatctacacagctccctcgggtctggagtccacttgtttggttgtggcctatggtttggacatttaccaaactcgagtctacccatccaagcagtttgacgttctgaaggatgactatgactacgntgtaatcagcagcgtcctctttgcctggtttttgcaccatgatcactaagagactgcacaggtgaaagctctggatcgggcttgcgataaagaacaagactgtgcctaaagtgggagccagggagtgtgggtaaatacaagtcacgttgagtttgtggattgtggagattgggggggaaggctaactaaaactggggaagatgtgacctcaccaaactctt

SEQ ID NO: 16 - DNA, 717 bp, artificial - cDNA prey sequence
  misc_feature (569)..(585): ny nucleotide
tggacagatagtctgattacagaacaactaaggtaataagaagaccaaggagaggccgcatgggtgtgcgaagagatgagccaaaggtgaaatctcttgcggatcacgagtggaatagaactcaacagattggagtactaagcagccacccttttgaaagtgacactgaaatgtctgatattgatgatgatgacagagaaacaatttttagctcaatggatcttctctctccaagtggtcattccgatgcccagacgctagccatgatgcttcaggaacaattggatgccatcaacaaagaaatcaggctaattcaggaagaaaaagaatctacagagttgcgtgctgaagaaattgaaaatagagtggctagtgtgagcctcgaaggcctgaatttggcaagggtccacccaggtacctccattactgcctctgttacagcttcatcgctggccagttcatcttcccccagtggacactcaactccaaagctcaccccctcgaagccctgccagggaaatggattcgatgggagtcatgacacttgcaagggatctgaggaaacatcngagaaaggatgccaanttttggaagaagatggttcggaagacaaagcaacaattaaatgtgaaacttttcctcttctaccccttaagccttaaaagggataaacttttctttttctaacccaagaagctgaaagagttaattttcttt

SEQ ID NO: 17 - DNA, 696 bp, artificial - cDNA prey sequence
  misc_feature (312)..(312): n = any nucleotide
agtgcggcctgggcacccgctgcctctgctcttgcctgcctgtgggcatcaccatgccccgatgcctgactacagctgcctgaagccacccaaggcaggcgaggaagggcacgagggctgctcctacaccatgtgccccgaaggcaggtatgggcatccagggtaccctgccctggtgacatacagctatggaggagcagttcccagttactgcccagcatatggccgtgtgcctcatagctgtggctctccaggagagggcagagggtatcccagccctggtgcccactccccacgggctggctccatttncccgggcaggccggcctatccacaatctaggaaagctgaggctacgaagatcccttacggagggagggagggggacagggaaccccattggcctggggcaacctggaccttaagcaaggaaccttttggcaatctgccagaagtccgcttggagccccggtgtccctgggaagggaaggggccccccaaatgggggaacaaagaacaaatgtgctttgggggctttcccccgagaaggccccccaatgccagggggttttcgttaaagaagtggggtttggggcccctttcacagccccctttgacaaaccaaaaaagtccacatccccagggggaaagggaaaagaccccctgggagaaaggggaaaaccccggggccccccc

SEQ ID NO: 18 - DNA, 724 bp, artificial - cDNA prey sequence
  misc_feature (512)..(512): n = any nucleotide
gtggagcgtgagtggcgttacgagtgtgacgggtctgaagatgatgccaatgtaaaagggtgcatgaatggggacgagataattcctgggccataatcagcatacctaatcacagttgagggtaaaaaacacatcttgatcatagagggagcaacaaaggctgatgctgcagaatattcagtaatgacaacaggaggacaatcatctgctaaacttagtgttgacttgaaacctctgaagattttgacacctctgactgatcagactgtaaatcttggaaaagaaatctgcctgaagtgtgaaatctctgaaaacataccaggaaaatggactaaaaatggcctacctgttcaggagagtgaccgtctaaaggtggttcagaagggaaggatccacaagttagtgatagccaatgccctcactgaagatgaaggtgattatgtatttgcacctgatgcctacaatgttactctgcctgccaaagttatggtattgattcttctaagatcatnctggattgtcttgatgctgacaacaccatgacggtgattgcaggaaacagcttcgtcttgagattcccattagcggagaaccacttcctaaaccatttggaagccgggaagtaaggttctattgaaaggcatggcccggttaaaaaccgaattttaacttggttgacccacttctggcattgattatactgaagggtgacttctggttttac

SEQ ID NO: 19 - DNA, 594 bp, artificial - cDNA prey sequence
acattgaaagaaatgccttggggacatatcaataacaacgtaacacagagctattctattggttatgaaggtagctatgatgcctctgctgatctctttgatgatattgctaaagaaatggacattgcaactgagattaccaaaaaatcacaggatattttgttaaaatggggaacatctttggcagaaagtcacccttcagagtctgatttttcactgagatcactttctgaagacttcatccagccttcacaaaaattatccttgcaaagcctatctgactctaggcattcaagaacatgctctccaacacctcattttcaatcagattcagaatataattttgaaaatagtcaagactttgtccatgttcacagtcaacttcaatttcagggttcaccaaacaagaattcatgggataaacagagctttaaaaaaacctgattttatcagatcttgatgtaactattaaaaaataaggattttccttaaaatgacaaccacaagccacccaactggccaaaaatttaaaacacttaccggaaataagaggcaatccaccactggcggccttcaggatcatttaagagccac

SEQ ID NO: 20 - DNA, 275 bp, artificial - cDNA prey sequence
aatatacaacatggctcgagcccatgcctgcaggcgccacgtctgcacaagagagagatgacgacatcatatggacatccacactcgcaaagcaggtcaggaggactggcatgcccctgtctccccagcaccccatttgtagccttttctcaggttgagtaaatagttctgtattaggaaaggccctcttgcctccacaactccttccccaccttggtgacatcattcatcgtggttctgccacttcctaggagcccatggaggagaggcaccaa

SEQ ID NO: 21 - DNA, 866 bp, artificial - cDNA prey sequence
  misc_feature (6)..(18): n=any nucleotide
aggggnnnnncccttttnttatcctcctacttgaggatgtgcgaaattatgcctctgacgaattggaacgaggggctaggcgtagattatggcggtctgtcaaatctacttggggagcagctaattctggacgagttagccggcctgctgcgaggccgctcataaagctgggactccatgacttacatcacttccactcccttgccatccgaggtgacatgcccaatcagattgtgcagatcttgacccaggatcatggcatggaattaatatgttgctttggcaacaccagttgggacagaagccttctgctcttcagggcaaaacaaaccatagagactcatccaatccctgaatcactgatagaaaaagggaaagaaaagaacagattaagattccagaagcagtgagatgggagggcangaagaccaagaaagatattgaaaggttttatattgagaaatatgttcattcttcttaattcctaacaatcangcagccgcaaaacctgcaggagcttttggtaaaatgtccaaggcacaatattggaaagaatcataatctggtccccaatggttttgaaccaaaccttgaagaagaagtgaaatcgtggggaggtgaatgagaccctagggaaatctctggaaatggggaaaagggcccatagggaaaaaaggggggcccccgggttatatggggtttatatgggaaaagaggtctttccttttttttggggggtatatttttttttttaaaggagatccaacccccgggctctggggcttttaaaaaaaaaattttggggaggttccccgggggccctcctccttaaaaaaccccacccccccggggttttttttcaaggc

SEQ ID NO: 22 - DNA, 552 bp, artificial - cDNA prey sequence
  misc_feature (4)..(4): n = any nucleotide
aggnggccatatacagtatcagtgctttcctggttataagctccatggaaattcatcaagaaggtgcctctccaatggctcctggagcggcagctcaccttcctgcctgccttgcagatgttccacaccagtaattgaatatggaactgtcaatgggacagattatgactgtggaaaggcagcccggattcagtgcttcaaaggcttcacgctcctaggactgtctgaaatcacctgtgaagccgatggccagtggagctctgtgttcccccactgtgaacacacttcatgtggttctcttccaatgataccaaatgcggtcatctcttctcggaagggcctggccatcctgaaactatgcaaagaaatccaacatgcgctgggcctttgtaagtaaacctgtaccttgagttactttttttattagggggaataaattgggaattccttggaaaaaaattattaaatggtgcattttaaaaaatcgcgggttttccttttaaaaattttttaattggagctgccttaccttaaaaaaaaatgaaatgtggg

SEQ ID NO: 23 - DNA, 783 bp, artificial - cDNA prey sequence
  misc_feature (34)..(109): n = any nucleotide
gaaatggccccttccccttgaaccctctgtcatnngtataggtngncntcatgataattcagtcgacatcggntcgccatctnangatctgnggacatctgcacgcgcngaggatacacagtgcagaacacatgtggcgggcacgcgtactgagatccgcacactagcagccaaaagctcattaccgccccgcatagttgcatagtcatcttagcaggagccgccatcatgtaacatacatatcgtgaacgcttacattcaccgcattgacacttacataaaagatcccaaagaaagggaatttctcttcaatgccattgaaacgatgccttgtgtcaagaagaaggcagactgggccttgcgctggattgnggacaaagaggctacctatggtgaacgtgttgtagcctttgctgcagtggaaggcattttcttttccggtcttttgcgtcgatattctggctcaagaaacgaggactgatgcctgcctcacatttctaatggactatatagcgagaatagggttacactgggaatttgcttgcctgaggtccaaacacccggccccaaaccttcggngggaaagtaggagaataatttcatgctgtccgtatgaacacggatcctaactgaggcttgcctgtaacgtaattggatgattgccctcaatgagcatactttggttttggcaacaacattcctgaaccggtttaccagtttcaaaaaaaacctttgcttttggaatttccctgaggaactacttttgaaaagaggccgttaaaa

SEQ ID NO: 24 - DNA, 833 bp, artificial - cDNA prey sequence
  misc_feature (6)..(57): n = any nucleotide
aggggntcttttnnnnngatgcctcctaccctgatgatggtgcgcagattagtcgcncgtgtgacgagatctggacatatcgcacggcgcatggcgcccaacgcatagcaggacgctcgcagaagcagcatgagccccggctcacattcccgcgcgaagaacatgcgtaaccaacagcgtgtctggaccacagccctgtcaccctgacactgaatcgcacgcaatgctagctgcccctttcccgtcctgggcaccccgagtctcccccgaccccgggtcccaggtatgctcccacctccacctgccccactcaccacctctgctagttccagacacctccacgcccacctggtcctctcccatcgcccacaaaagggggggcacgagggacgagcttagctgagctgggaggagcagggtgagggtgggcgacccaggattccccctccccttcccaaataaagatgagggtactaaaanaaaaaaaaaaaanaaaannnnccccagaaaggtttgggttttttccccaaggggttgggaaagattccaaaaaaggggtggcgtggtgtgaaaannnnnnaaaaccnnnngnaatngaaccctttgttattcaaaagcttgttgggaaaaggaaaaccccccccctttgaactaacaatttttaaaattgaactgttactaaacagaaaaaaaagtttttggttttttttgatctgactgtaatgaaaannnnattttttcctagggttttaaagagtaatactttttgtaaaactctttggaagtgggcctttggaaaggaaaaaattgttttntagggaaactatttaaag

SEQ ID NO: 25 - DNA, 639 bp, artificial - cDNA prey sequence
  misc_feature (498)..(498): n=any nucleotide
aaggactacccagtggtgtctatcgaagatccctttgaccaggatgactggggagcttggcgagaagttcacagccagtgcaggaatccaggtagtgggggatgatctcacagtgaccaacccaaagaggatcgccaaggccgtgaacgagaagtcctgcaactgcctcctgctcaaagtcaaccagattggctccgtgaccgagtctcttcaggcgtgcaagctggcccaggccaatggttggggcgtcatggtgtctcatcgttcgggggagactgaagataccttcatcgctgacctggttgtggggctgtgcactgtgcagatcaagactggtgccccttgccgatctgagcgcttggccaagtacaaccagctcctcagaattgaagaggagctgggcagcaaggctaagtttgccggcaggaacttcagaaaccccttgccaagtaagctgtgggcaggcaagcccttcggtcacctgttgtctacacagancccttccctcgtgtcagctcaggcagctcgaggccnncgaccaacacttgcaggggtccnttgctagtagcgccccacccgcgtggagttcgtaccgcttctttagacttcntacagaagccaagcttccttggagccctg

SEQ ID NO: 26 - DNA, 760 bp, artificial - cDNA prey sequence
  misc_feature (533)..(533): n = any nucleotide
ggatgcctcctacctctgatgatgtgccataattagtcacctgtcacggattcgaatcgagcgcggacgagtcgaccatgctgtgcgcgcgaggcgaccagcgggcgctctaacagccgcctgatcgcggacctgttgagcgccgactaagactagacgttattgaccactcacgtgaacctactagcccacaggcggttttgtgagctgcttccccaggagcagcggagcgtggagtcgtcacttcgggcacaagtgcccttcgagcagattctcagccttccagagctcaaggccaaccccttcaaggagcgaatctgcagggtcttctccacatccccagccaaagacagccttagctttgaggacttcctggatctcctcagtgtgttcagtgacacagccacgccagacatcaagtcccattatgccttccgcatctttgactttgatgatgacggaaccttgaacagagaagacctgagccggctggtgaactgcctcacgggagagggcgaggacacacggcttantgcgtctgagatgaagcaactcatcgacaacattctggaggagtctgacattgacaggatggaccatcaactctctgagtncagcacgtnatctcccgtcttcagactttgcaagttctttagaatgcctgtgacagaaccccagctgggtctggaccttgtcaaaacctttactgtgactttggcaagtaaacttgttgcaatgcggccacttggcaactgactgg

SEQ ID NO: 27 - DNA, 644 bp, artificial - cDNA prey sequence
  misc_feature (505)..(505): n = any nucleotide
tgtggacctcgtcgatgaacagcactccttcctcaaccgggccctggagagtgacatggcgcctgtcctgatcatggccaccaaccgtggcatcacgcgaatccggggcaccagctaccagagccctcacggcatccccatagacctgctggaccggctgcttatcgtctccaccaccccctacagcgagaaagacacgaagcagatcctccgcatccggtgcgaggaagaagatgtggagatgagtgaggacgcctacacggtgctgacccgcatcgggctggagacgtcactgcgctacgccatccagctcatcacagctgccagcttggtgtgccggaaacgcaagggtacagaagtgcaggtggatgacatcaagcgggtctactcactcttcctggacgagtcccgctccacgcagtacatgaaggagtaccaggacgccttcctcttcaacgaactcaaaggcgagaccatggacacctcctgagttggatgtcatccnccgaccccaccctgttttccaccagagttctgacactgtgactctgtataaaatgggtgggaagctgcacccaccctgtgtatgtgtggttgccctgagcccncngaatgccanaaaataaaaaataattccttagaag

SEQ ID NO: 28 - DNA, 636 bp, artificial - cDNA prey sequence
  misc_feature (513)..(513): n = any nucleotide
aagacgcagctgacatcaatgctcatgatgaacctggaatccaggcctgtgatcttcgaggatgtggggaggcaggtgctggccactcgctccagaaagctgccgcacgagctgtgcacgctcatccgcaacgtgaagccggaagatgtgaagagagtcgcttctaagatgctccgagggaagccggcagtggccgccctgggtgacctgactgacctgcccacgtatgagcacatccagaccgccctgtcgagtaaggacgggcgcctgcccaggacgtaccggctcttccggtagaaccgctccccggcctgacagacccagggagctgcagctggagcccgttcccgtgcgtgttagtttgtacacgaatttagtctaaaaagctgtctggttgtataaacggtgcaaacaatgtcgccacagcacccacgcggattgcattcttttggaactcaatgtgccgatcagtggagtcagtatcgagcctgaccaccgcaagccaggaagcangtgaagtgcccagcgctggagtgcatcgtgccacgaggagggcggtcggtgcttcccttctcgagctgtgggcacatagcgccccgcaggttccttggatgtagccctgatctaggtagcacc

SEQ ID NO: 29 - DNA, 860 bp, artificial - cDNA prey sequence
  misc_feature (8)..(8): n = any nucleotide
ctcttttntttatcctcctacttgatgatgtgcgaaatcagtaccgctgacgaactgggaactgagcggcggatactggagtggcatcgacaagtcgaatcgaggtcgcaccaagcggcgacagctgataaccatcacgaacagccttgcatcattgagcaccgcatcactgccaacagttgtaggcacgactaacatccactcgcaagggcagaaggttgaagaacaggagcctgaactgacatcaactcccaatttcgtggttgaagttataaagaatgatgatggcaagaaggcccttgtgttggactgtcattatccagaggatgaggttggacaagaagacgaggctgagagtgacatcttctctatcagggaagttagctttcagtccactggcgagtctgaatggaggatactaattatacactcaacacagantccttggactggcccttatatgaccaccctatgaatttccttgccgaccgagggggtgacaacacttttgccagataaccggtggaactcagcccaagccttgagcaacagggagtccattacttttcttggagaaccttaggaaattttgtcaagagagccctttaaacccccaccaatgcctgaaaagcccttagttttcaatgggcagggcctttggccccaggggaacaaaaaaccctcaccctttaaaagctttaacaactgggcccttttggaaaaggggagttttcaaccccccaaaatcccaaaagggggggaaaaaaaacccccccaattttaaaaaatttttttgggtttggggggggggcccccaatattaaaataaaaaaattttttttttctgttgacacaaaaaa